(19) United States
(12) Patent Application Publication
(10) Pub. No.: US 2012/0282647 A1
(43) Pub. Date: Nov. 8, 2012

(54) ASSAY FOR QUANTIFYING CLOSTRIDIAL NEUROTOXIN

(75) Inventors: Gerd J Mander, Frankfurt Am Main (DE); Harold Victor Taylor, Frankfurt Am Main (DE); Martin Vey, Wayne, PA (US); Karl-Heinz Eisele, Frankfurt Am Main (DE)

(73) Assignee: MERZ PHARMA GmbH & CO.KGaA, FRANKFURT am MAIN (DE)

(21) Appl. No.: 13/509,404

(22) PCT Filed: Nov. 16, 2010
(86) PCT No.: PCT/EP2010/006967

§ 371 Date: Jul. 27, 2012

(30) Foreign Application Priority Data
Nov. 18, 2009 (EP) PCT/EP2009/008214

(51) Int. Cl. C12Q 1/02

(52) U.S. Cl. 435/29

(57) ABSTRACT

Method of measuring an effect induced to a muscle tissue by a clostridial neurotoxin, comprising:
(a) contacting a muscle tissue or a cell culture with a sample comprising the clostridial neurotoxin;
(c) measuring the effect induced to the muscle tissue by the clostridial neurotoxin;
   wherein step (c) is performed in the absence of the sample.

### FIELD OF THE INVENTION

This invention relates to an ex vivo method for determining the unknown concentration of a clostridial neurotoxin in a sample with respect to the known concentration of a clostridial toxin in a reference sample. The method may comprise electrically stimulating muscle tissues that have been contacted with said samples and comparing the respective effects induced to said muscle tissues, thereby determining said unknown concentration. The method can also be used to estimate the relative potency of a clostridial neurotoxin in a sample with respect to a reference standard.

### BACKGROUND OF THE INVENTION

In recent years, botulinum neurotoxins have become the standard agent in the treatment of focal dystonias and spastic indications. Pharmaceutical preparations are commercially available e.g. by Ipsen Ltd. (Dysport®) or Allergan Inc. (Botox®). A high purity neurotoxin, free of any other clostridial proteins is e.g. available from Merz Pharmaceuticals (Xeomin®). Another preparation was registered by Solstice Neurosciences, Inc. (Myobloc®). Still another preparation was registered by Mentor Corporation (PurTox®). These preparations either differ in the used botulinum toxin type or in the biological efficacy, respectively the potency.

Treatment of patients generally involves injection of the neurotoxin into affected muscle tissue, bringing the agent near the neuromuscular end plate, i.e. close to the cellular receptor mediating its uptake into the nerve cell controlling said affected muscle. Various degrees of neurotoxin spread have been observed. This spread is thought to correlate with the injected amounts and the particular preparation of neurotoxin injected. Resulting from the spread, systematic side effects caused by the inhibition of acetylcholine release may be observed at nearby muscle tissue. The incidents of unintended paralysis of untreated muscles can largely be avoided by reducing the injected doses to the therapeutically relevant level. Overdosing may also be a problem with regard to the patients' immune system, as the injected neurotoxin may trigger the formation of neutralizing antibodies. If this occurs, the toxin will be inactivated without being able to relieve the involuntary muscle activity.

Discrepancy on the dose equivalents or variations in the determined potency of preparations such as available sales products or batches produced during the manufacturing process poses an increased risk for patients through possible side effects and the development of immunity. Therefore, it is of crucial importance to determine the concentration of clostridial neurotoxin contained in said sales products or production batches reliably (i.e. without significant variation) and as accurately as possible, in order to adjust the toxin concentration to a reliable effective dose for the benefit of the patient. This may also serve as an incentive to the manufacturers to offer formulations allowing optimum exploitation of biological activity, i.e. potency, for different therapeutic purposes.

EP 1 597 584 B1 suggests an ex vivo method for determining the quantity of a pre-synaptic neuromuscular blocking substance in a sample, such as a sample containing botulinum neurotoxin. The method comprises electrically stimulating a muscle tissue, preferably the rib muscle of a mouse, in the presence of the sample containing the pre-synaptic neuromuscular blocking substance and comparing the effect induced by the sample to the effect induced by a reference substance and thereby determining the quantity of the pre-synaptic neuromuscular blocking substance in the sample.

GB 2 416 849 A and GB 2 398 636 A suggest an ex vivo method for determining the quantity of a pre-synaptic neuromuscular blocking substance in a sample, such as a sample containing botulinum neurotoxin. The method comprises electrically stimulating a smooth muscle tissue, preferably the rib muscle of a mouse or a rat, in the presence of the sample containing the pre-synaptic neuromuscular blocking substance and comparing the effect induced by the sample to the effect induced by a reference substance and thereby determining the quantity of the pre-synaptic neuromuscular blocking substance in the sample.

US 2003/0032891 A1 suggests an in vivo method for measuring potency of a substance such as a clostridial toxin, wherein said substance is administered to a mammal, the mammal is subjected to a stimulus and the pinna reflex response of said mammal to said stimulus is monitored.

EP 2 015 065 A1 suggests a method for quantifying the efficacy of a neurotoxin such as a Clostridium neurotoxin, wherein said toxin is administered to the hind leg of a non-human mammal, an electrical stimulus is applied to said non-human mammal and the contraction of said hind leg is measured and is compared to the contraction of the other hind leg.

Pearce et al., Toxicon, Vol. 35, No. 9, pp. 1373-1412, 1997, disclose the suitability of the rat/mouse phrenic nerve-hemidiaphragm for binding botulinum neurotoxin.

Wohlfahrt et al., Naunyn-Schmiedeberg's Arch Pharmacol 355, 335-340 (1997) compare the efficacy of two commercial botulinum toxin A preparations by dose dependent response curves by using mouse diaphragm.

Chang et al., Naunyn-Schmiedeberg's Arch. Pharmacol. 282, 129-142 (1974) compare presynaptic actions of type A botulinum toxin and β-bungarotoxin on isolated nerve-muscle preparations such as mouse and rat diaphragms.

Sheridan et al., J. Appl. Toxicol. 19, S29-S33 (1999) describe the determination of the efficacy of botulinum antagonists based on classical bioassays of toxin concentration.

James et al., Am. J. Physiol. Gastrointest. Liver Physiol. 285, G291-G297 (2003) describe inhibitory effects of botulinum toxin on pyloric and antral smooth muscle.

Göschel et al., Exp. Neurol., vol. 147, 1, 1997 describe concentration-response curves for determining the relative potency of botulinum toxin in a sample compared to the potency of a sample containing a known amount of toxin. Different botulinum toxin preparations were tested on mouse hemidiaphragms.

The above referenced prior art quantification methods, however, lack the precision required for certification by regulatory authorities. Therefore, the methods disclosed therein cannot be used for regulatory purposes, instead still an out-of-time mouse killing assay must be performed.

### OBJECTS OF THE INVENTION

One object of the invention is to improve the methods of the prior art and to develop a reliable and accurate method for determining the potency, respectively the concentration of a clostridial neurotoxin in a sample effecting said potency, and which might be used for regulatory purposes. Such an improved method would also serve to satisfy the great need for a safe and effective administration.

### SUMMARY OF THE INVENTION

In one aspect, the invention relates to a method of measuring an effect induced to a muscle tissue by a clostridial neurotoxin, comprising:
(a) contacting a muscle tissue with a sample comprising said clostridial neurotoxin;
(c) measuring said effect induced to said muscle tissue by said clostridial neurotoxin;

wherein step (c) is performed in the absence of said sample.

In one embodiment, said muscle tissue is electrically stimulated.

In one embodiment, the method comprises step (b) subsequent to step (a):
(b) electrically stimulating said muscle tissue obtained in step (a).

In another embodiment, step (b) is performed in the absence of said sample.

In another aspect, the invention relates to a method of determining the unknown concentration of a clostridial neurotoxin in a first sample with respect to the known concentration of a clostridial neurotoxin in a second sample, the method comprising:
(a) contacting a muscle tissue with said second sample;
(c) measuring a second effect induced to said muscle tissue by said neurotoxin;
(d) repeating steps (a) to (c) at various concentrations of said clostridial neurotoxin;
(e) recording said measured second effect of step (d) versus concentration, thereby recording a second data set;
(f) contacting a muscle tissue with said first sample;
(h) measuring a first effect induced to said muscle tissue;
(k) identifying the concentration for which said first and said second effect are identical;
(l) equating said concentration in (k) to said unknown concentration.
wherein step (c) and/or step (h) is/are performed in the absence of said second and/or first sample.

In one embodiment, said muscle tissue is electrically stimulated.

In one embodiment, the method comprises step (b) subsequent to step (a) and step (g) subsequent to step (f):
(b) electrically stimulating said muscle tissue obtained in step (a);
(g) electrically stimulating said muscle tissue obtained in step (f).

In another aspect, the invention relates to a method of determining the relative potency of a clostridial neurotoxin in a first sample with respect to the potency of clostridial neurotoxin in a second sample, the method comprising:
(a) contacting a muscle tissue with said second sample;
(c) measuring a second effect induced to said muscle tissue by said neurotoxin;
(d) repeating steps (a) to (c) at various concentrations of said clostridial neurotoxin;
(e) recording said measured second effect of step (d) versus concentration, thereby recording a second data set;
(f) contacting a muscle tissue with said first sample;
(h) measuring a first effect induced to said muscle tissue;
(i) repeating steps (f) to (h) at various concentrations of said clostridial neurotoxin;
(j) recording said measured first effect of step (i) versus concentration, thereby recording a first data set;
wherein step (c) and/or step (h) is/are performed in the absence of said second and/or first sample.

In one embodiment, said muscle tissue is electrically stimulated.

In one embodiment, the method comprises step (b) subsequent to step (a) and step (g) subsequent to step (f):
(b) electrically stimulating said muscle tissue obtained in step (a);
(g) electrically stimulating said muscle tissue obtained in step (f).

In one embodiment, the method further comprises steps (m) and (n):
(m) selecting said various concentrations from a concentration range that best fits to the first and the second data set;
(n) determining said best fit by a statistical test comprising the following sub-steps (α) to (δ):
(α) representing a value range of the second data set obtained in step (e) by a fit curve;
(β) representing a value range of the first data set obtained in step (j) by a fit curve;
(γ) linearizing the fit curves, respectively;
(δ) parallelizing the linearized fit curves.

In one embodiment, the statistical test is a F-test, or a x²-test, or a t-test.

In one embodiment, the false-rejection probability for each sub-step (α) to (δ) is ≦5 (expressed in %).

In one embodiment, the method further comprises step (ε):
(ε) calculating from the shift of the linearized and parallelized fit curves relative to each other the relative potency of the first sample with respect to the second sample.

In one embodiment of the invention according to the methods of the second and third aspect, steps (b) or (g) are performed in the absence of the second or the first sample, or steps (b) and (g) are performed in the absence of the second and the first sample.

In one embodiment according to any one of the methods of the three aspects according to the invention, the period of exposure of the muscle tissue to said clostridial neurotoxin, i.e. the period of contacting a muscle tissue with a sample, respectively a first or a second sample comprising a clostridial neurotoxin, according to step (a) prior to the absence of said sample, respectively the first or the second sample, respectively the measuring of said effect according to step (c) or step (h), or step (c) and step (h), is from 1 to 60 min.

In one embodiment according to any one of the methods of the three aspects according to the invention, prior to said measuring in step (c) or step (h), or step (c) and step (h), said muscle tissue is exposed to said clostridial toxin for a period of from 5 to 30 min.

In a further embodiment according to any one of the methods of the three aspects according to the invention, the period of exposure of said muscle tissue to said neurotoxin is approximately 15 minutes.

In one embodiment according to any one of the methods of the three aspects according to the invention, said muscle tissue is already electrically stimulated prior to step (a) and/or step (f).

In another embodiment, said muscle tissue is already electrically stimulated during step (a) and/or step (f).

In another embodiment, said muscle tissue is already electrically stimulated prior to step (a) and during step (a) and/or prior to step (f) and during step (f).

In one embodiment, said recording of said measured second effect is performed by plotting said second effect versus concentration, and said recording of said second data set is performed by recording a calibration curve.

In one embodiment, said second effect is determined at at least one concentration expressed in mouse LD₅₀ units/ml of at least 10.

In another embodiment, said concentration is from 10 to 1,000, or from 10 to 70, or from 15 to 60, or from 20 to 45.

In another embodiment, said concentration is from 20 to 400, or is from 100 to 800.

In one embodiment, said mouse LD₅₀ units are Xeomin® units.

In one embodiment, said effect, respectively said first and second effects, are selected from the group consisting of time to paralysis of said muscle tissue, variation in the contraction rate of said muscle tissue, variation in the contraction distance of said muscle tissue, variation in the force of contraction of said muscle tissue, variation in the end plate potential or the miniature end plate potential of said muscle tissue.

In one embodiment, said effect, respectively said first and second effect, is the time to paralysis.

In one embodiment, said muscle tissue is selected from intercostal muscle, hind limb muscle and the hind limb extensor digitorum longus muscle, the plantar muscles of the hind paw, the phrenic nerve-hemidiaphragm, the levator auris longus muscle, the frog neuromuscular junction, the biventer cervic muscle of chicks, rib muscles, brain tissue or the electrical organ of the sea ray.

In one embodiment, said phrenic nerve-hemidiaphragm is of rat or mouse.

In one embodiment, said clostridial neurotoxin is botulinum toxin.

In another embodiment, said botulinum neurotoxin is of a serotype selected from the group consisting of A, B, C, D, E, F and G; or is a chemically or genetically modified derivative of a botulinum neurotoxin of a serotype selected from the group consisting of A, B, C, D, E, F and G.

In one embodiment, the neurotoxin is free of complexing proteins.

In another embodiment, said neurotoxin is of serotype A or B.

In one embodiment, said electrical stimulation is performed in a buffer comprising an anti-foaming agent.

In one embodiment, said anti-foaming agent is selected from silicon-containing compounds.

In one embodiment, said buffer is purged with oxygen.

In another aspect, the invention relates to a computer program product comprising a computer program comprising software means for implementing the method according to the invention.

In another aspect, the invention relates to a kit comprising:

(A)
a device for stimulating a muscle tissue that has been exposed to a clostridial neurotoxin to select an effect induced by said neurotoxin to said muscle tissue;
a device for measuring and recording said effect; and
(B) a computer program product comprising a computer program comprising software means for implementing the method according to the invention.

In another aspect, the invention relates to the use of a muscle tissue in any one of the methods of the invention.

In another aspect, the invention relates to the use of the method of the invention according to any one of the three aspects of the invention for controlling the potency of a sample comprising a clostridial neurotoxin.

In one embodiment, the sample is a stored sample.

In one embodiment, the sample is a lyophilized sample or is a reconstituted sample.

In one aspect, the invention relates to the use of the method according to the first aspect of the invention for determining the unknown concentration of a clostridial neurotoxin in a first sample with respect to the known concentration of a clostridial neurotoxin in a second sample; or for determining the relative potency of a clostridial neurotoxin in a first sample with respect to the potency of a clostridial neurotoxin in a second sample, e.g. during the quality control during a process for the manufacture of clostridial neurotoxin.

### DETAILED DESCRIPTION OF THE INVENTION

It has been found that the variability observed with the quantification methods of the prior art can be reduced significantly to an insignificant degree by applying the methods disclosed herein.

According to a first aspect, the invention relates to a method for measuring an effect induced to a muscle tissue by a clostridial neurotoxin, comprising:
(a) contacting a muscle tissue with a sample comprising said clostridial neurotoxin;
(c) measuring an effect induced to said muscle tissue by said neurotoxin;

wherein step (c) is performed in the absence of said sample.

The term “contacting a muscle tissue with said sample (that may be a first or a second sample according to the methods according to the further aspects of the invention)” means that at least part of said neurotoxin of said sample is received by said muscle tissue during said contacting, i.e. at least part of the neurotoxin being contained in said sample is bound by appropriate receptors being contained in said muscle tissue.

The term “absence of the sample” means that the measuring of the effect in step (c) is performed in a medium, typically an appropriate buffer, that contains 10% by weight or less, e.g. does not contain any, of the sample or, stated differently, of the neurotoxin of the sample.

In one embodiment, said muscle tissue is not continuously exposed to the sample (that may be a first or a second sample according to the methods according to the further aspects of the invention) comprising a clostridial neurotoxin, but only temporarily.

This means that after a predetermined period of exposing said muscle tissue to the neurotoxin, i.e. contacting in step (a) in order to effect a response of said muscle tissue to the exposure, the corresponding measurement of the effect (or a first, respectively second effect according to the methods according to the further aspects of the invention), wherein e.g. said muscle tissue is electrically stimulated, is performed in the absence of said sample (that may be said first or said second sample according to the methods according to the further aspects of the invention) employing the methods as described below.

In one embodiment, prior to said measurement, said muscle tissue is e.g. removed from an organ bath containing said sample, and is transferred to an organ bath containing the neurotoxin-free ingredients as described below. Subsequently, the electrical stimulation and the measurement of the magnitude of the effect (that may be a first or a second effect when the sample is a first or a second sample) is performed. This means that the electrical stimulation and the response to said stimulation is performed with the muscle tissue containing the received neurotoxin.

In another embodiment, the neurotoxin-containing ingredients, i.e. the sample (that may be a first or a second sample), are replaced by neurotoxin-free ingredients. Subsequent to the replacement, the measurement of the magnitude of the effect (that may be a first or a second effect when the sample is a first or a second sample) is performed.

The term “clostridial neurotoxin (or clostridial toxin)” encompasses clostridial toxin complexes as well as high purity neurotoxin, i.e. a neurotoxin preparation, which is free of any other clostridial proteins.

In one embodiment, said clostridial neurotoxin is botulinum neurotoxin.

In another embodiment, said botulinum neurotoxin is a serotype selected from the group consisting of A, B, C, D, E, F and G.

The term “botulinum toxin complex” encompasses a botulinum toxin associated with at least another non-toxic protein. As apparent, the term botulinum toxin complex, as used herein, comprises the 450 kDa and the 900 kDa botulinum toxin complex, which is e.g. obtainable from cultures of C. botulinum. Such preparations on the basis of botulinum toxin complex of type A are commercially available e.g. by Ipsen Ltd. (Dysport®) or Allergan Inc. (Botox®). Another preparation based on botulinum complex type B is available from Solstice Neurosciences, Inc. (Myobloc®). A high purity neurotoxin of type A, free of any other clostridial proteins is available from Merz Pharmaceuticals (Xeomin®). It is the drug of choice to improve several forms of focal dystonia.

In another embodiment, said botulinum neurotoxin is a chemically or genetically modified derivative of a serotype selected from the group consisting of A, B, C, D, E, F and G.

A chemically modified derivative of said neurotoxin may be one that is modified by pyruvation, phosphorylation, sulfatation, lipidation, and/or glycosilation.

A genetically modified derivative of said neurotoxin is one that has been modified by deletion, addition or substitution of one or more amino acids contained in the proteins of said serotype.

Such a modified toxin preferably is biologically active.

A biologically active toxin is a toxin being capable to be uptaken into a cell, thereby proteolytically cleaving one or more polypeptides involved in the SNARE complex.

In one embodiment, said muscle tissue is electrically stimulated.

In one embodiment, the method further comprises:
(b) electrically stimulating said muscle tissue obtained in step (a).

In one embodiment, step (b) is performed in the absence of said sample.

Surprisingly, it has been discovered that the electrical stimulation and measurement of said effect in the absence of said sample, after said muscle tissue had been exposed to the neurotoxin, shifts the respective dose-response curves such that the sensitivity of the method according to the invention is significantly increased. The sensitivity is particularly increased at low concentrations expressed in LD₅₀ mouse units/ml of said clostridial neurotoxin in said sample.

For example, if as effect, respectively response, the time to paralysis is determined, said time to paralysis is increased compared to a method, wherein said effect is measured in the presence of the sample. This results in an advantageous increase of the sensitivity of the method, which in particularly applies in the region of lower concentrations of neurotoxin. If the potency is determined at lower concentration, neurotoxins in general may exhibit the greatest variances, whereas at rather high concentrations potencies converge to each other.

This increasing of the sensitivity allows for a more precise and more reliable analysis of the respective dose-response curves. This in turn allows for a considerably lower amount of laboratory animals such as mice, which otherwise have to be sacrificed in order to perform any one of the methods according to the invention. Accordingly, this embodiment of the invention is not only a progress under technical aspects but also under ethical aspects.

The term “sensitivity” is used herein in the meaning as commonly used in physiology, i.e., it defines the ability of a muscle tissue to respond to an external stimuli. Here, the external stimuli is performed by contacting a muscle tissue with a clostridial neurotoxin. It is within the ambit of the invention that a certain concentration range may be chosen, such as a concentration range at relatively low concentration of clostridial neurotoxin, where said sensitivity is increased, i.e. a response can be determined that otherwise can not be determined, respectively can only determined within a non-tolerable deviation.

According to a second aspect, the invention relates to a method for determining the unknown concentration of a clostridial neurotoxin in a first sample with respect to the known concentration of a clostridial neurotoxin in a second sample, the method comprising:
(a) contacting a muscle tissue with said second sample;
(c) measuring a second effect induced to said muscle tissue by said neurotoxin;
(d) repeating steps (a) to (c) at various concentrations of said clostridial neurotoxin;
(e) recording said measured second effect of step (d) versus concentration, thereby recording a second data set;
(f) contacting a muscle tissue with said first sample;
(h) measuring a first effect induced to said muscle tissue;
(k) identifying the concentration for which said first and said second effect are identical;
(l) equating said concentration in (k) to said unknown concentration.
wherein step (c) and/or step (h) is/are performed in the absence of said second and/or first sample.

In one embodiment, said muscle tissue is electrically stimulated.

In one embodiment, the method comprises step (b) subsequent to step (a), or step (b) subsequent to step (a) and step (g) subsequent to step (f):
(b) electrically stimulating said muscle tissue obtained in step (a);
(g) electrically stimulating said muscle tissue obtained in step (f).

In another embodiment, steps (b) or (g) are performed in the absence of the second or the first sample, or steps (b) and (g) are performed in the absence of the second and the first sample.

Accordingly, in one embodiment, the determination of the second and/or the first effect is performed in the absence of said second and/or first sample.

In another embodiment, the electrical stimulation of said muscle tissue is performed in the absence of said second and/or first sample. This means that after step (a) and prior to step (b) and/or after step (f) and prior to step (g) said muscle tissue is removed from the second and/or the first sample as disclosed above.

The term “identifying the concentration for which said first and said second effect are identical” (steps (k) and (l)) means that said first and second effect are qualitatively and quantitatively identical, i.e. the induced effect is e.g. the time to paralysis, and that said effects have the same measured value.

In one embodiment, in order to obtain results that can reliably be compared, the exposure time of the muscle tissue to the neurotoxin being contained in the second, respectively the first sample, should be comparable.

In one embodiment, said exposure times are identical.

In one embodiment, said recording of said measured second effect in step (e) is performed by measuring said second effect at various concentrations of said clostridial neurotoxin in said second sample and plotting said measured second effect versus concentration, thereby recording a calibration curve.

If the effect induced by said second sample to said muscle tissue is determined on the basis of various concentrations expressed in mouse LD₅₀ units/ml, a calibration curve may be obtained, as described above.

For example, it is possible, to determine said effect induced in steps of ten LD₅₀ units/ml or of five LD₅₀ units/ml within a selected concentration range.

Accordingly, by means of the second data set recorded in step (e), a calibration curve is plotted by means of which the unknown concentration of said clostridial neurotoxin in said first sample is identified according to steps (k) and subsequent step (l).

In one embodiment, the generated calibration curve is plotted, and said steps of identifying and equating according to steps (k) to (l), are performed by a graphic analysis.

Said unknown concentration of the first sample can be determined by identifying the concentration from the calibration curve for which said first and said second effect have the same value, e.g. the same time to paralysis, and equating said concentration to said unknown concentration according to step (l).

A prerequisite for said determination is that the unknown concentration of the clostridial toxin in the first sample exerts an effect on the muscle tissue, which can be quantified by means of said calibration curve. The person skilled in the art will readily acknowledge that it may be necessary to dilute or concentrate the first sample having the unknown concentration once or several times if necessary in order to achieve a concentration range, wherein a comparison with the second sample is possible, i.e. to achieve identical first and second effects. Then, knowing the dilution or the concentration factor, the calculation of the concentration of the neurotoxin being originally present in the not diluted or not concentrated first sample may be determined.

In another embodiment, said identification and equation is not performed by a single-point measurement of only one concentration in step (h) and subsequent steps (k) and (l), but by measurement at a multitude of various concentrations. This is particularly important in view of regulatory requirements.

According to another aspect of the invention, it is desirable to optimize the concentration range in which a reliable comparison of said second and first sample is possible. This does not only apply to the comparability regarding the biological efficacy of hitherto known and commercial formulations of clostridial neurotoxins, but also to formulations, which might by developed in future or being already under development.

In one embodiment, in order to optimize the concentration range expressed in mouse LD₅₀ units/ml in which a reliable comparison of said second and first sample is possible, it is desirable to firstly determine the standard deviation of the calibration curve recorded in step (e) and/or in step (h). By using a suitable step-wise regression analysis, it is possible to generate a regression model for predicting the potency of an unknown toxin sample based on the dose-response curve.

By means of such method, it is possible to identify a concentration range for the first and the second sample representing two different data populations, in which the correlation between the respective dose-response curves reaches a maximum, i.e. the best fit is determined.

In one embodiment, the test may be further refined by representing a value range of the respective data sets of the first and the second sample by fit curves according to a predetermined regression model, respectively, and linearizing and parallelizing said fit curves within a predetermined confidence interval.

Accordingly, according to a third aspect, the invention relates to a method of determining the relative potency of a clostridial neurotoxin in a first sample with respect to the potency of clostridial neurotoxin in a second sample, the method comprising:
(a) contacting a muscle tissue with said second sample;
(b) electrically stimulating said muscle tissue obtained in step (a);
(c) measuring a second effect induced to said muscle tissue by said neurotoxin;
(d) repeating steps (a) to (c) at various concentrations of said clostridial neurotoxin;
(e) recording said measured second effect of step (d) versus concentration, thereby recording a second data set;
(f) contacting a muscle tissue with said first sample;
(g) electrically stimulating said muscle tissue obtained in step (f);
(h) measuring a first effect induced to said muscle tissue obtained in step (g);
(i) repeating steps (f) to (h) at various concentrations of said clostridial neurotoxin;
(j) recording said measured first effect of step (i) versus concentration, thereby recording a first data set;
wherein step (c) and/or step (h) is/are performed in the absence of said second and/or first sample.

In one embodiment, the method further comprises steps (m) and (n):
(m) selecting said various concentrations from a concentration range that best fits to the first and the second data set;
(n) determining said best fit by a statistical test comprising the following sub-steps (α) to (δ):
(α) representing a value range of the second data set obtained in step (e) by a fit curve;
(β) representing a value range of the first data set obtained in step (j) by a fit curve;
(γ) linearizing the fit curves, respectively;
(δ) parallelizing the linearized fit curves.

In one embodiment, said muscle tissue is electrically stimulated.

In one embodiment, the method comprises step (b) subsequent to step (a) and step (g) subsequent to step (f):
(b) electrically stimulating said muscle tissue obtained in step (a);
(g) electrically stimulating said muscle tissue obtained in step (f).

In one embodiment, steps (b) or (g) are performed in the absence of the second or the first sample, or steps (b) and (g) are performed in the absence of the second and the first sample.

Accordingly, in one embodiment, the determination of the second and/or the first effect is performed in the absence of said second and/or first sample.

In another embodiment, the electrical stimulation of said muscle tissue is performed in the absence of said second and/or first sample. This means that after step (a) and prior to step (b) and/or after step (f) and prior to step (g) said muscle tissue is removed from the second and/or the first sample as disclosed above.

Statistical tests suitable for performing the above sequence are well known, such as likelihood-quotient-tests. An example of such a likelihood-quotient-test is the known F-test. Test such as the x²-Test (chi-squared-test or x²-distribution-test) or the t-test may also be employed. Said tests are also known in the art.

In one embodiment, said statistical test is the F-test.

By means of said test, it is possible to decide within a predetermined confidence interval whether two random samples taken from two different populations essentially differ with respect to the variance thereof. Therefore, such a test serves for the testing of differences within two statistical samples, here the second and the first sample.

In one embodiment, the confidence interval should be broad in order to obtain reliable results, i.e. the false-rejection probability should be relatively low.

In one embodiment, the false-rejection probability is ≦5 (expressed in %; (or 0.05)), respectively the confidence interval is ≧95 (expressed in %; (or 0.95)).

In one embodiment, the false-rejection probability for each sub-step (α) to (δ) is ≦5 (expressed in %).

In one embodiment, linearizing in step (γ) is performed by representing the respective data sets by a best fit straight line.

In one embodiment, parallelizing in step (δ) is performed by determining a common slope of the best fit straight lines.

Subsequent to step (δ), from the shift of the linearized and parallelized fit curves relative to each other, the relative potency of the first sample versus the second sample is determined.

Accordingly, in one embodiment, the method further comprises after step (δ) step (ε):
(ε) calculating from the shift of the linearized and parallelized fit curves relative to each other the relative potency of the first sample with respect to the second sample.

In one embodiment, the term “relative potency” means that the potency of the first sample with respect to the second sample is determined at identical concentration, respectively identical concentrations, from the respective linearized and parallellized fit curves.

In one embodiment, the potency of the second sample is equated to 100%, and the relative potency of the first sample is expressed in terms of %. E.g., one obtains for the first sample a potency of e.g. 110% or 90% with respect to the second sample. By respective dilution of the first sample having the 110% potency to the 100% potency, one obtains the effective concentration of the clostridial neurotoxin in the first sample, which hitherto was not known, by application of the rule of three. The unit for measurement now becomes relative potency, and the value is expressed as a unit of activity (potency) defined in terms of the activity (potency) of the reference standard (second sample).

In another embodiment, the relative potency is expressed as ratio of the potency of the first and the second sample.

In one embodiment, the above described model is used to predict the logarithmic value of the applied neurotoxin dose.

In another embodiment, both the quantity of the stimulated effect and the quantity of the neurotoxin dose in the sample are recorded in a logarithmic scale.

In one embodiment, the second effect, respectively the first effect, are measured at at least three different concentrations of the clostridial neurotoxin in the second sample, respectively the first sample.

In one embodiment, said recording of said date sets, respectively said recording of a calibration curve, respectively calibration curves, is performed in the form of a semi logarithmic plot.

In another embodiment, a double logarithmic plot is performed.

The method of determining a relative potency is documented in the European Pharmacopoeia.

In one embodiment, starting with a concentration of e.g. 10 mouse LD₅₀ units/ml, the method of determining said relative potency is applied over the whole range of the data set. Subsequently, values greater than 10 mouse LD₅₀ units/ml are used as starting points, such as 11, 12, 13, 14, 15, 16, 17 mouse LD₅₀ units/ml. Said iteration is performed as long until the applied model yields the desired and required accuracy.

In one embodiment, once a best fit and thus a concentration range has been identified by the statistical test, any first sample having an unknown concentration (with regard to the effective concentration) of a clostridial neurotoxin may be compared with respect to the known concentration of said clostridial neurotoxin in a second sample within said concentration range identified according to the method of the invention.

In one embodiment, said recording of said measured second effect is performed by plotting said second effect versus concentration, and said recording of said second data set is performed by recording a calibration curve.

The use of relative potency estimates, and the inclusion of a reference standard (second sample) in the assay, lead to more precise and more reproducible estimates, which provide opportunities for reductions in animal use.

In one embodiment according to any one of the methods according to the three aspects according to the invention, prior to said measuring in step (c) or step (h) or step (c) and step (h), said muscle tissue is exposed to said clostridial toxin for a period of from 5 to 30 min.

In one embodiment according to any one of the methods according to the three aspects according to the invention, said muscle tissue is already electrically stimulated prior to step (a) and/or step (f).

In another embodiment, said muscle tissue is already electrically stimulated during step (a) and/or step (f).

In another embodiment, said muscle tissue is already electrically stimulated prior to step (a) and during step (a) and/or prior to step (f) and during step (f).

Statistical tests are commonly performed by means of a suitable computer program and a suitable computer.

In one embodiment, the statistical test is performed by means of a suitable computer program comprising suitable software means for implementing the statistical test.

Accordingly, in one embodiment, the invention relates to a computer program product comprising a computer program comprising software means for implementing the method according to the invention.

In one embodiment, the second sample is selected from a commercially available and registered botulinum toxin preparation. Since these products are registered and allowed as a pharmaceutical preparation, respectively medicament, they comprise a clearly defined quantity, respectively concentration of a botulinum toxin.

In another embodiment, any botulinum toxin preparation may be used that has been produced under standard conditions.

In one embodiment, the commercial preparations mentioned above may be used as the second sample. Thus, the second sample may be Xeomin®, Botox®, Dysport®, Myobloc® or PurTox®. These preparations either differ in the used botulinum toxin type or in biological efficacy/activity, i.e. potency, e.g. in the concentration of the botulinum neurotoxin or in the botulinum type contained therein.

The mouse unit expressed in terms of mouse LD₅₀ is a commonly accepted unit to define a concentration of a clostridial neurotoxin contained in a sample. The LD₅₀ value defines the lethal dose at which 50% of a mouse population is killed if said quantity is applied to the mice of said mouse population. The method for determining said value is known to the person skilled in the art. Such method is documented in the European Pharmacopoeia.

As is known, the LD₅₀ units in the labeling of the products based on a botulinum neurotoxin may be product-specific, respectively manufacturer-specific, and may be non-interchangeable due to the absence of a standard.

In one embodiment, the LD₅₀ units referred to herein are units as determined in the characterization and labeling of Xeomin®. E.g., the second sample is Xeomin®. Accordingly, the units relating to a certain potency are Xeomin® units. Therefore, the assay system of the present invention can be used for comparably assessing the potency of any sample comprising a clostridial neurotoxin relative to Xeomin®. Then, the method allows to directly compare first samples comprising a clostridial neurotoxin (in an unknown concentration) in terms of Xeomin® units.

Xeomin® and Botox® exhibit an approximately comparable efficacy or potency. In order to obtain the same efficacy or potency as Xeomin® and Botox®, approximately the 2.5-fold quantity of Dysport®, respectively the 10-fold quantity of Myobloc® have to be applied.

In one embodiment, these commercially available preparations are diluted or concentrated to predetermined concentrations of the botulinum neurotoxin contained therein, and said second effect is measured in dependence of various concentrations of said clostridial neurotoxin in said second sample. Said measured effect is plotted versus concentration of botulinum toxin, thereby recording a calibration curve. By means of said second data set, respectively said calibration curve, the unknown concentration of botulinum neurotoxin in a first example may be determined.

It has been discovered that a concentration of a clostridial neurotoxin in a sample (that may be a first or a second sample) expressed in mouse LD₅₀ units/ml of at least 10, the methods according to the invention can be advantageously applied. It is to be noted that the concentration given within the present application are all mouse LD₅₀ units/ml.

In one embodiment, said concentration is at least 15.

In another embodiment, said concentration is at least 20.

In another embodiment, said concentration is from 10 to 1,000.

In one embodiment, the concentration is from 10 to 70.

In another embodiment, the concentration is from 15 to 60.

In still another embodiment, the concentration is from 20 to 45.

In one embodiment, the second sample is Xeomin®.

In one embodiment, it has been discovered that if Xeomin® is used as the second sample, particularly reliable results are obtained, if the second effect is determined at at least one concentration of from 10 to 70. In another embodiment, the concentration is from 15 to 60. In still another embodiment, the concentration is from 25 to 45.

In one embodiment, it has been discovered that if Botox® is used as the second sample, reliable results are obtained, if the second effect is determined at at least one concentration of from 10 to 70. In another embodiment, the concentration is from 15 to 60. In still another embodiment, the concentration is from 25 to 45.

If a second sample is used for determining the calibration curve according to step (e), the second sample having a lower concentration or comprising a less efficient or potent botulinum neurotoxin than Xeomin® or Botox®, higher concentrations of the neurotoxin, i.e. higher LD₅₀ units/ml values are required in order to achieve a strength of the second effect that is comparable to the effect induced by Xeomin® or Botox®.

In an embodiment, wherein the second sample has a lower concentration or potency of botulinum neurotoxin than Xeomin® or Botox®, the second effect is determined at at least one concentration of from 20 to 400, or from 100 to 800.

In one embodiment, wherein the second sample is Dysport®, the second effect is determined at at least one concentration of from 20 to 400, or from 25 to 300, or from 30 to 250.

In another embodiment, wherein the second sample is Myobloc®, the second effect is determined at at least one concentration of from 100 to 800, or from 150 to 700, or from 200 to 600.

In other embodiments, the concentration may range from 30 to 600, or 30 to 400, or 30 to 200, or 30 to 100, or 30 to 80, or 40 to 500, or 40 to 400, or 40 to 300, or 40 to 200, or 40 to 100, or 40 to 90, or 50 to 300, or 50 to 200, or 50 to 100, or 60 to 100, depending on the concentration of the efficacy or potency of the neurotoxin in the second sample compared to Xeomin® or Botox®.

In one embodiment, the LD₅₀ units are Xeomin® units.

According to a first variant of the invention, the effect used to determine said unknown concentration is the time to paralysis of a muscle tissue. Time may be measured e.g. in seconds or minutes. According to sub-variants, the time to paralysis may be determined based on the muscle contraction distance (paralysis being achieved once the contraction distance is equal to 0), or on the muscle twitch frequency (paralysis being achieved once the twitch frequency is equal to 0). The contraction distance may e.g. be measured in centimeters or millimeters.

The “time to paralysis” may be defined as the period that passed to attain half maximum twitch. This is strictly dependent on the toxin concentration.

According to other variants of the invention, the effect induced is the variation in the contraction rate of the muscle tissue, or is the variation in the contraction of the muscle tissue, or is the variation in the force of contraction of the muscle tissue, or is the variation in the end plate potential or the miniature end plate potential of the muscle tissue. These methods are known in the art, and are e.g. disclosed in EP 1 597 584 B1.

In one embodiment, the effect, respectively the first and second effect induced, is the time to paralysis of the muscle tissue.

Basically, any muscle tissue can be selected for the method of the invention that exhibits neuromuscular characteristics, that is which responds to an electrical stimulation. By muscle tissue is meant a preparation comprising one or more muscle fibers having a nerve cell or nerve cells or a nerve attached thereto, which may be electrically stimulated. Both smooth and striated muscle tissue can be used.

According to the teaching of the present invention, muscular tissue comprises the intercostal muscle, the hind limb muscle and the hind limb extensor digitorum longus muscle e.g. of mice and rats, the plantar muscles of the hind paw e.g. of the mouse or rat, the phrenic nerve-hemidiaphragm e.g. of the rat or mouse, the levator auris longus muscle e.g. of the mouse and rat, the frog neuromuscular junction, the biventer cervic muscle of chicks. Rib muscles or brain tissue e.g. of the mouse and rat or the electrical organ of the sea ray may also be used.

Moreover, in one embodiment, experiments have shown that using the mouse phrenic nerve-hemidiaphragm is a suitable tool for measuring clostridial toxicity. Thus, it may be used as an assay for determining clostridial toxicity.

In one embodiment, due to the reliability of said mouse hemidiaphragm assay, it is possible to comply with certification requirements of regulatory authorities and to satisfy the need for a safe and effective administration of botulinum toxin such as of serotype A or serotype B.

In one embodiment, the hemidiaphragm is a hemidiaphragm of a rodent, such as rat or mouse.

In one embodiment, the hemidiaphragm is the mouse hemidiaphragm.

The term “mouse or rat hemidiaphragm” means the phrenic nerve-hemidiaphragm of the rat or mouse.

In still another embodiment, said clostridial toxin in said first sample and said clostridial toxin in said second sample are the same clostridial toxins.

In still another embodiment, said clostridial toxin or neurotoxin in the first sample and said clostridial toxin or neurotoxin in said second sample are different from each other.

For the experimental realization of the method, typically muscle tissue with attached motor neurons is removed from an animal such as a mouse or rat, and is placed in an organ or tissue bath containing a buffer such as a physiologic buffer, in which conditions such as ionic composition, glucose, temperature, pH and oxygenation are controlled to optimize tissue viability and performance. Measurements of the force of muscle contraction following electrical stimulation can be made when the muscle is attached to a force transducer, and this affords a direct measure of the effect of toxin on neuromuscular function.

In one embodiment, the temperature in the buffer is from 35 to 39° C., or from 36 to 38° C. In another embodiment, the temperature is from 36.5 to 37.5° C.

In still another embodiment, the temperature is or is approximately 37° C.

In one embodiment, said pH in said buffer is from 7 to 8, or from 7.2 to 7.8. In one embodiment, said pH is or is approximately 7.5.

In one embodiment, oxygenation is performed with a gas mixture comprising oxygen. In one embodiment, oxygenation is performed with a mixture of carbon dioxide and oxygen. In one embodiment, a gas mixture consisting of 95 parts oxygen (based on volume) and 5 parts carbon dioxide (based on volume) is employed. Commercially available mixtures are known as carbogene.

For carrying out the electrical stimulation in order to measure an effect, respectively a second and first effect, basically the methods of the referenced prior art may be used.

In one embodiment, the method is carried out such that the electrical stimulation in step (b) or (g), is carried out at a voltage at least equal to the supra-maximal voltage. By supra-maximal voltage is understood the minimum voltage to get the maximum twitch response of the muscle tissue. In general, such an experiment is repeated several times, and the results are averaged in order to obtain a reliable result.

The electrical stimulation may be carried out such that at a voltage at least equal to the supra-maximal voltage of said tissue is stimulated at certain time intervals by pulse stimulation. By pulse stimulation is meant stimulations lasting a certain time separated from each other by periods lasting a time during which no stimulation is exerted. This approach is disclosed e.g. in Göschel et al., Exp. Neurol., vol. 147, 1, 1997, Wohlfahrt et al., Naunyn-Schmiedeberg's Arch Pharmacol (1997) 355:335-340.

Alternatively, the electrical stimulation may be train pulse stimulation. Such a method is disclosed in EP 1 597 584 B1.

In one embodiment of the pulse stimulation, the duration of the stimulations may range from 10 μs to 1 ms. The duration of the periods in which no stimulation is exerted may range from 0.1 to 10 s. The supra-maximal voltage may range between e.g. 1 mV and 15 V. The muscle tissue is e.g. continuously electro-stimulated with pulses at a frequency of e.g. 1 Hz via two electrodes.

Microelectrodes may be placed at or near the neuromuscular junctions and intracellular recordings of spontaneous and evoked membrane potentials can be recorded. These membrane potentials are produced by the activation of ligand-gated ion channels by acetylcholine, which in turn are influenced by the toxin. Analysis of the endplate potentials may be used to obtain information about the effect of toxin on quantal release of acetylcholine.

Specifically, a suitable muscle tissue, e.g. the left phrenic nerve-hemidiaphragm (nervus phrenicus) may be excised e.g. from a male or female mouse and placed in an organ bath. In one embodiment, this organ bath is a bath containing Krebs-Ringer-Solution, or Earle's Balanced Salt Solution (EBSS), or physiological saline. Said solutions are known to the person skilled in the art. The muscle tissue is then stimulated via the nerve phrenicus in the presence of the first respectively the second sample according to the known methods. The induced effects are recorded and evaluated also employing known methods, e.g. the methods as described in the referenced prior art.

The muscle tissue may be immersed in a buffer, such as a physiological buffer. The buffer may comprise an energy source. The energy source may be an ATP energy source, e.g. one or more of the following: ATP, a sugar such as glucose and/or creatine, a fatty acid, an amino acid, glycogen, a surfactant and pyruvic acid.

The buffer may be oxygenated, particularly for longer assays. Preferably, oxygen and glucose (or other ATP source) may be added to the organ bath in order to extend life span of said muscle tissue. Adding a surfactant may be beneficial in particular to reduce bubbles, which may have a negative impact on the method of the invention.

In one embodiment, the surfactant is an anti-foaming agent.

The term “anti-foaming agent” comprises all agents that affect the surface tension of gas bubbles, which are embedded in a liquid.

One type of anti-foaming agents lowers the surface tension of gas bubbles, which are embedded in a liquid, thereby breaking the gas bubbles.

However, it is also possible that anti-foaming agents may increase the surface tension of gas bubbles with the effect that said bubbles coalesce to larger bubbles, which escape from the liquid easier than small bubbles.

The affection of the surface tension may be measured by methods that are known to the person skilled in the art, such as contact angle and wetting angle measurements.

Therefore, an anti-foaming agent is an agent that prevents formation of foam or breaks foam already formed.

Commonly used anti-foaming agents are insoluble oils, dimethyl polysiloxanes and other silicones, alcohols, stearates and glycols.

In one embodiment, the anti-foaming agent is selected from at least one silicon-containing compound.

In a further embodiment, at least one silicon-containing compound is a siloxane.

The term “siloxane” comprises oligosiloxanes and polysiloxanes. In one embodiment, said siloxanes are substituted with alkyl groups and/or aryl groups. Such siloxanes are well known in the art. It is possible to apply silicon-containing compounds in the form of an individual compound or in the form of a mixture of more than one silicon-containing compounds.

Examples of suitable silicon compounds, respectively suitable siloxanes, but not limited thereto, are α-(trimethylsilyl)-ω-methylpoly[oxy(dimethylsilylen)] and polydimethylsiloxane. Such compounds are commercially available and are used in or as medicaments, e.g. under the names simethicone and dimethicone.

The person skilled in the art will readily acknowledge that other compounds having a similar activity such as dimethicone and simethicone can also be applied in the method of the present invention.

In another aspect, the invention relates to a kit comprising an organ bath, in which the muscle tissue is stimulated that has been exposed to a clostridial neurotoxin, and wherein the effect of said stimulation is measured (e.g. as described above), and a computer program product by means of which the statistical test is performed, thereby optimizing the concentration range in which the effect generated by the neurotoxin is to be measured in order to obtain reliable results.

Accordingly, in one embodiment, the invention relates to a kit comprising:
(A) a device for stimulating a muscle tissue that has been exposed to a clostridial neurotoxin to select an effect induced by said neurotoxin to said muscle tissue;
   a device for measuring and recording said effect; and
(B) a computer program product comprising a computer program comprising software means for implementing the method according to the invention.

According to a fourth aspect, the invention also provides an improved method of identifying a concentration range in which in which the potency of a first sample comprising a clostridial neurotoxin relative to a second sample comprising a clostridial neurotoxin can be determined within a predetermined confidence interval or false-rejection probability.

In one embodiment, such method of identifying a concentration range in which the potency of a first sample comprising a clostridial neurotoxin relative to a second sample comprising a clostridial neurotoxin may be determined, comprises the following steps:
(a) contacting a muscle tissue with said second sample;
(b) electrically stimulating said muscle tissue obtained in step (a);
(c) measuring a second effect induced to said muscle tissue by said neurotoxin;
(d) repeating steps (a) to (c) at various concentrations of said clostridial neurotoxin;
(e) recording said measured second effect of step (d) versus concentration, thereby recording a second data set;
(f) contacting a muscle tissue with said first sample;
(g) electrically stimulating said muscle tissue obtained in step (f);
(h) measuring a first effect induced to said muscle tissue by said neurotoxin;
(i) repeating steps (f) to (h) at various concentrations of said clsotridial neurotoxin;
(j) recording said measured first effect of step (i) versus concentration, thereby recording a first data set;
wherein said concentration is selected from a concentration range that best fits to the first and the second data set, and wherein said best fit is determined by a statistical test comprising the following sub-steps (α) to (δ):
(α) representing a value range of the second data set obtained in step (e) by a fit curve;
(β) representing a value range of the first data set obtained in step (j) by a fit curve;
(γ) linearizing the fit curves, respectively;
(δ) parallelizing the linearized fit curves.

In said embodiment, said second and said first effect are qualitatively identical. For refining the method, the methods as described above in connection with the method according to the third aspect of the invention can be used.

In a further aspect of the invention, the methods of the invention may be advantageously used for controlling the quality, i.e. the potency of a sample comprising a clostridial neurotoxin with respect to a reference standard such as is required in a manufacturing process.

Accordingly, in said aspect, the invention relates to the use of the method of the invention for controlling the quality, i.e. the potency of a sample comprising a clostridial neurotoxin.

In one embodiment, the potency of a sample is determined that has been stored. In one embodiment, the sample has been stored for a period of at least one hour, or at least one day.

In one embodiment, the sample is a lyophilized sample, or is a reconstituted sample.

According to another aspect, the invention relates to the use of the method according to the first aspect of the invention for determining the unknown concentration of a clostridial neurotoxin in a first sample with respect to the known concentration of a clostridial neurotoxin in a second sample; or for determining the relative potency of a clostridial neurotoxin in a first sample with respect to the potency of a clostridial neurotoxin in a second sample.

According to a further aspect, the invention relates to the use of a muscle tissue, in particular a mouse or rat hemidiaphragm, for determining clostridial activity in any one of the methods of the invention, or for determining clostridial activity by aid of the kit according to the invention.

The following embodiments also belong to the invention and are to be understood that the embodiments described above apply vice versa to the methods listed below.

Thus, the invention also relates to an ex vivo method for determining an unknown concentration of a clostridial neurotoxin in a first sample with respect to the known concentration of a clostridial neurotoxin in a second sample, the method comprising:
(a) contacting a muscle tissue with said second sample;
(b) electrically stimulating said muscle tissue obtained in step (a);
(c) measuring a second effect induced to said muscle tissue by said neurotoxin;
(d) repeating steps (a) to (c) at various concentrations of said clostridial neurotoxin;
(e) recording said measured second effect of step (d) versus concentration, thereby recording a second data set;
(f) contacting a muscle tissue with said first sample;
(g) electrically stimulating said muscle tissue obtained in step (f);
(h) measuring a first effect induced to said muscle tissue obtained in step (g);
   wherein said second effect is determined at at least one concentration expressed in mouse LD₅₀ units/ml of at least 10.

In one embodiment, the concentration is identified for which said first and said second effect are identical, and is equated to the unknown concentration of said clostridial neurotoxin in said first sample.

Accordingly, in one embodiment, the method further comprises steps (k) and (l):
(k) identifying the concentration for which said first and said second effect are identical;
(l) equating said concentration in (k) to said unknown concentration.

In one embodiment, said muscle tissue is already electrically stimulated prior to step (a) and/or step (f).

In another embodiment, said muscle tissue is already electrically stimulated during step (a) and/or step (f).

In another embodiment, said muscle tissue is already electrically stimulated prior to step (a) and during step (a) and/or prior to step (f) and during step (f).

Said electrical stimulation of said muscle tissue may be performed in the absence or presence of the second and/or the first sample, provided said muscle tissue has been exposed to said clostridial neurotoxin being present in said second and/or first sample.

In one embodiment, the invention relates to an ex vivo method for determining an unknown concentration of a clostridial neurotoxin in a first sample with respect to the known concentration of a clostridial neurotoxin in a second sample, the method comprising:
(i) electrically stimulating a muscle tissue in the presence of said second sample and selecting a second effect induced by said second sample to said muscle tissue,
(ii) measuring said second effect in (i) at various concentrations of said clostridial neurotoxin in said second sample and plotting said measured second effect versus concentration, thereby recording a second data set,
(iii) electrically stimulating said muscle tissue in the presence of said first sample,
(iv) selecting a first effect induced by said first sample to said muscle tissue,
(v) identifying the concentration for which said first and said second effect are identical, and
(vi) equating said concentration in (v) to said unknown concentration,
wherein said second effect is determined at at least one concentration expressed in mouse LD₅₀ units/ml of at least 10.

In one embodiment, said recording of said measured second effect in step (e) or step (ii) is performed by measuring said second effect at various concentrations of said clostridial neurotoxin in said second sample and plotting said measured second effect versus concentration, thereby recording a calibration curve.

Accordingly, by means of the second data set recorded in step (e) or (ii), a calibration curve is plotted by means of which the unknown concentration of said clostridial neurotoxin in said first sample is identified according to steps (k) and subsequent step (l), respectively step (v) and subsequent step (vi).

In one embodiment, the generated calibration curve is plotted, and said steps of identifying and equating according to steps (k) to (l), respectively step (v) and subsequent step (vi), are performed by a graphic analysis.

In one embodiment, said concentration is at least 15, or is at least 20.

In another embodiment, said concentration is from 10 to 1,000.

In one embodiment, the concentration of the second sample is from 10 to 70.

In another embodiment, the concentration of the second sample is from 15 to 60.

In still another embodiment, the concentration is from 20 to 45.

In one embodiment, the commercial preparations mentioned above may be used as the second sample. Thus, the second sample may be Xeomin®, Botox®, Dysport®, Myobloc® or PurTox®.

In one embodiment, the used units are Xeomin® units.

In one embodiment, these commercially available preparations are diluted or concentrated to predetermined concentrations of the botulinum neurotoxin contained therein, and said second effect is measured in dependence of various concentrations of said clostridial neurotoxin in said second sample. Said measured effect is plotted versus concentration of botulinum toxin, thereby recording a calibration curve. By means of said second data set, respectively said calibration curve, the unknown concentration of botulinum neurotoxin in a first example is determined.

In one embodiment, it has been discovered that if Xeomin® is used as the second sample, particularly reliable results are obtained, if the second effect is determined at at least one concentration of from 10 to 70. In another embodiment, the concentration is from 15 to 60. In still another embodiment, the concentration is from 25 to 45.

In one embodiment, it has been discovered that if Botox® is used as the second sample, reliable results are obtained, if the second effect is determined at at least one concentration of from 10 to 70. In another embodiment, the concentration is from 15 to 60. In still another embodiment, the concentration is from 25 to 45.

If a second sample is used for determining the calibration curve according to step (ii), the second sample having a lower concentration or comprising a less efficient or potent botulinum neurotoxin than Xeomin® or Botox®, higher concentrations of the neurotoxin, i.e. higher LD₅₀ units/ml values are required in order to achieve a strength of the second effect that is comparable to the effect induced by Xeomin® or Botox®.

In an embodiment, wherein the second sample has a lower concentration or potency of botulinum neurotoxin than Xeomin® or Botox®, the second effect is determined at at least one concentration of from 20 to 400, or from 100 to 800.

In one embodiment, wherein the second sample is Dysport®, the second effect is determined at at least one concentration of from 20 to 400, or from 25 to 300, or from 30 to 250.

In another embodiment, wherein the second sample is Myobloc®, the second effect is determined at at least one concentration of from 100 to 800, or from 150 to 700, or from 200 to 600.

In other embodiments, the concentration may range from 30 to 600, or 30 to 400, or 30 to 200, or 30 to 100, or 30 to 80, or 40 to 500, or 40 to 400, or 40 to 300, or 40 to 200, or 40 to 100, or 40 to 90, or 50 to 300, or 50 to 200, or 50 to 100, or 60 to 100, depending on the concentration of the efficacy or potency of the neurotoxin in the second sample compared to Xeomin® or Botox®.

If the effect induced by said second sample to said muscle tissue is determined on the basis of various concentrations expressed in mouse LD₅₀ units/ml, a calibration curve may be obtained, as described above.

For example, it is possible, to determine said effect induced in steps of ten LD₅₀ units/ml or of five LD₅₀ units/ml within the indicated concentration ranges.

Said unknown concentration of the first sample can be determined by identifying the concentration from the calibration curve for which said first and said second effect have the same value, e.g. the same time to paralysis, and equating said concentration to said unknown concentration according to step (l).

A prerequisite for said determination is that the unknown concentration of the clostridial toxin in the first sample exerts an effect on the muscle tissue which can be quantified by means of said calibration curve. The person skilled in the art will readily acknowledge that it may be necessary to dilute or concentrate the first sample having the unknown concentration once or several times if necessary in order to achieve a concentration range, wherein a comparison with the second sample is possible, i.e. to achieve identical first and second effects. Then, knowing the dilution or the concentration factor, the calculation of the concentration of the neurotoxin being originally present in the not diluted or not concentrated sample may be determined.

In one embodiment, the method is carried out such that the electrical stimulation in step (b) or (g), respectively (i) and (iii), is carried out at a voltage at least equal to the supra-maximal voltage employing the methods of the prior art as described above.

In one embodiment, the muscle tissue is the mouse diaphragm.

Accordingly, the method for determining the unknown concentration of a clostridial neurotoxin in a first sample with respect to the known concentration of a clostridial neurotoxin in a second sample comprises:
(i) electrically stimulating a mouse hemidiaphragm in the presence of said second sample and selecting a second effect induced by said second sample to said mouse hemidiaphragm,
(ii) measuring said second effect in (i) at various concentrations of said clostridial neurotoxin in said second sample and plotting said measured second effect versus concentration, thereby recording a calibration curve,
(iii) electrically stimulating said muscle tissue in the presence of said first sample,
(iv) measuring a first effect induced by said first sample to said muscle tissue,
(v) identifying the concentration for which said first and said second effect are identical, and
(vi) equating said concentration in (v) to said unknown concentration,
wherein said second effect is determined at at least one concentration expressed in mouse LD₅₀ units/ml of at least 10.

In one embodiment, said muscle tissue is the rat or mouse phrenic nerve-hemidiaphragm, the induced effect is the time to paralysis, and said clostridial botulinum is botulinum neurotoxin of serotype A.

In a specific embodiment of the invention, the method encompasses a method for determining the unknown concentration of botulinum neurotoxin of serotype A in a first sample with respect to the known concentration of a botulinum A toxin in a second sample, said method comprising:
(i) electrically stimulating a mouse hemidiaphragm in the presence of said second sample and selecting a second time to paralysis induced by said second sample to said mouse hemidiaphragm,
(ii) measuring said second effect in (i) at various concentrations of said clostridial neurotoxin in said second sample and plotting said measured second effect versus concentration, thereby recording a calibration curve,
(iii) electrically stimulating said muscle tissue in the presence of said first sample,
(iv) measuring a first effect induced by said first sample to said muscle tissue,
(v) identifying the concentration for which said first and said second effect are identical, and
(vi) equating said concentration in (v) to said unknown concentration,
wherein said second time to paralysis is determined at at least one concentration expressed in mouse LD₅₀ units/ml of from 10 to 70, or from 15 to 60, or from 20 to 45, and wherein the second sample is Xeomin® or Botox®.

In one embodiment, said concentration is in the range of from 16.6 mouse LD₅₀ units/ml to 56.3 mouse LD₅₀ units/ml.

In another embodiment, said concentration is in the range of from 20 mouse LD₅₀ units/ml to 55 mouse LD₅₀ units/ml.

In still another embodiment, said concentration is in the range of from 25 mouse LD₅₀ units/ml to 50 mouse LD₅₀ units/ml.

In another specific embodiment of the invention, the method encompasses a method for determining the unknown concentration of botulinum toxin of serotype A in a first sample with respect to the known concentration of a botulinum A toxin in a second sample, said method comprising:
(i) electrically stimulating a mouse hemidiaphragm in the presence of said second sample and selecting a second time to paralysis induced by said second sample to said mouse hemidiaphragm,
(ii) measuring said second effect in (i) at various concentrations of said clostridial neurotoxin in said second sample and plotting said measured second effect versus concentration, thereby recording a calibration curve,
(iii) electrically stimulating said muscle tissue in the presence of said first sample,
(iv) measuring a first effect induced by said first sample to said muscle tissue,
(v) identifying the concentration for which said first and said second effect are identical, and
(vi) equating said concentration in (v) to said unknown concentration,
wherein said second time to paralysis is determined at at least one concentration expressed in mouse LD₅₀ units/ml of from 20 to 400, or 25 to 300, or 30 to 250, and wherein the second sample is Dysport®.

In another specific embodiment of the invention, the method encompasses a method for determining the unknown concentration of botulinum neurotoxin of serotype B in a first sample with respect to the known concentration of a botulinum B toxin or a botulinum A toxin in a second sample, said method comprising:
(i) electrically stimulating a mouse hemidiaphragm in the presence of said second sample and selecting a second time to paralysis induced by said second sample to said mouse hemidiaphragm,
(ii) measuring said second effect in (i) at various concentrations of said clostridial neurotoxin in said second sample and plotting said measured second effect versus concentration, thereby recording a calibration curve,
(iii) electrically stimulating said muscle tissue in the presence of said first sample,
(iv) measuring a first effect induced by said first sample to said muscle tissue,
(v) identifying the concentration for which said first and said second effect are identical, and
(vi) equating said concentration in (v) to said unknown concentration,
wherein said second time to paralysis is determined at at least one concentration expressed in mouse LD₅₀ units/ml of from 100 to 800, or 150 to 700, or 200 to 600, and wherein the second sample is Myobloc®.

However, an assay for determining neurotoxin concentration or neurotoxin potency may not only be based on tissue as described in the foregoing, but also on cell cultures.

According to a further aspect, the invention relates to an assay for determining activity of clostridial neurotoxin based on cell cultures for determining the unknown concentration of a clostridial neurotoxin in a sample with respect to the known concentration of a clostridial toxin in a reference sample. The method makes use of the quantification of proteins such as SNAP25 resulting from the cleavage of a SNARE complex when cell cultures, which are sensitive for clostridial botulinum neurotoxin, are exposed to said neurotoxin. The method can also be used to estimate the relative potency of a clostridial neurotoxin in a sample with respect to a reference standard.

Pellet, S., et al., Comparison of the primary rat spinal cord cell (RSC) assay and the mouse bioassay for botulinum neurotoxin type A determination, Journal of Pharmacological and Toxicological Methods (2010), doi: 10.1016/j.vascn.2010.01.003, suggest a cell-based assay for potency determination of purified botulinum neurotoxin serotype A as an alternative to the mouse bioassay.

Keller, J. E., et al., Persistence of botulinum neurotoxin action in cultured spinal cord cells, FEBS Letters 456 (1999) 137-142, disclose the mechanism underlying the differences in persistence of botulinum neurotoxin A (BoNT/A) and botulinum neurotoxin E (BoNT/E) activities.

A further object of the invention is to improve these methods of the prior art and to develop a reliable and accurate method for determining the potency, respectively the concentration of a clostridial neurotoxin in a sample effecting said potency, and which might be used for regulatory purposes. Such an improved method would also serve to satisfy the great need for a safe and effective administration.

This further object is achieved by a method in which a cell culture is exposed to or contacted with a sample comprising a clostridial neurotoxin, wherein prior to the measurement of an effect, which is induced to the cells of the cell culture by said clostridial neurotoxin, said sample is replaced by an aqueous medium, such as a buffer, or such as a neutral buffer, which is free from a clostridial neurotoxin or said clostridial neurotoxin, and said cell culture is exposed to said aqueous medium for a defined period, e.g. a period of more than 1 hour, or more than 2 h, or more than 3 h, or more than 4 h, or more than 5 h. Prior to the measurement, the cell culture may be contacted with said aqueous medium for a period up to 100 h or even more.

Surprisingly, it has been discovered that the measurement of said effect in the absence of said sample, and subsequent to the contacting with an aqueous medium which is free from a clostridial botulinum neurotoxin after said cell culture had been exposed to or contacted with the sample comprising the neurotoxin, shifts the respective dose-response curves such that the sensitivity of the method according to the invention is significantly increased. The sensitivity is particularly increased at low concentrations expressed in LD₅₀ mouse units/ml of said clostridial neurotoxin in said sample.

Accordingly, in a first aspect, the invention relates to a method of measuring an effect induced to a cell culture by a clostridial neurotoxin, comprising:
(a) contacting a cell culture with a sample comprising said clostridial neurotoxin;
(c) measuring said effect induced to said cell culture by said clostridial neurotoxin;

wherein

step (c) is performed in the absence of said sample; and
prior to said measuring in step (c) and subsequent to the contacting in step (a), said cell culture is contacted for a period of from 0.5 to 100 h with an aqueous medium which is free from a clostridial toxin.

In a second aspect, the invention relates to a method of determining the unknown concentration of a clostridial neurotoxin in a first sample with respect to the known concentration of a clostridial neurotoxin in a second sample, the method comprising:
(a) contacting a cell culture with said second sample;
(c) measuring a second effect induced to said cell culture by said neurotoxin;
(d) repeating steps (a) to (c) at various concentrations of said clostridial neurotoxin;
(e) recording said measured second effect of step (d) versus concentration, thereby recording a second data set;
(f) contacting a cell culture with said first sample;
(h) measuring a first effect induced to said cell culture;
(k) identifying the concentration for which said first and said second effect are identical;
(l) equating said concentration in (k) to said unknown concentration.

wherein
step (c) and/or step (h) is/are performed in the absence of said second and/or first sample; and
prior to said measuring in step (c) or step (h) or step (c) and step (h) and subsequent to the contacting in step (a) or step (f) or step (a) and step (f), said cell culture is contacted for a period of from 0.5 to 100 h with an aqueous medium which is free from a clostridial toxin.

In a third aspect, the invention relates to a method of determining the relative potency of a clostridial neurotoxin in a first sample with respect to the potency of clostridial neurotoxin in a second sample, the method comprising:
(a) contacting a cell culture with said second sample;
(c) measuring a second effect induced to said cell culture by said neurotoxin;
(d) repeating steps (a) to (c) at various concentrations of said clostridial neurotoxin;
(e) recording said measured second effect of step (d) versus concentration, thereby recording a second data set;
(f) contacting a cell culture with said first sample;
(h) measuring a first effect induced to said cell culture;
(i) repeating steps (f) to (h) at various concentrations of said clostridial neurotoxin;
(j) recording said measured first effect of step (i) versus concentration, thereby recording a first data set;

wherein
step (c) and/or step (h) is/are performed in the absence of said second and/or first sample; and
prior to said measuring in step (c) or step (h) or step (c) and step (h) and subsequent to the contacting in step (a) or step (f) or step (a) and step (f), said cell culture is contacted for a period of from 0.5 to 100 h with an aqueous medium which is free from a clostridial toxin.

In one embodiment, the method further comprises steps (m) and (n):
(m) selecting said various concentrations from a concentration range that best fits to the first and the second data set;
(n) determining said best fit by a statistical test comprising the following sub-steps (α) to (δ):
   (α) representing a value range of the second data set obtained in step (e) by a fit curve;
   (β) representing a value range of the first data set obtained in step (j) by a fit curve;
   (γ) linearizing the fit curves, respectively;
   (δ) parallelizing the linearized fit curves.

In one embodiment, the statistical test is a F-test, or a x²-test, or a t-test.

In one embodiment, the false-rejection probability for each sub-step (α) to (δ) is ≦5 (expressed in %).

In one embodiment, the method further comprises step (ε):
(ε) calculating from the shift of the linearized and parallelized fit curves relative to each other the relative potency of the first sample with respect to the second sample.

In one embodiment, said effect (including the first and/or the second effect) is the cleavage of a protein from a SNARE complex.

In one embodiment, the protein is SNAP25.

In one embodiment, prior to said measuring in step (c) or step (h) or step (c) and step (h), said cell culture is contacted with said clostridial toxin for a period of from 5 to 45 h, or from 15 to 40 h, or from 25 to 35 h.

In one embodiment, prior to said measuring in step (c) or step (h) or step (c) and step (h) and subsequent to the contacting in step (a) or step (f) or step (a) and step (f), said cell culture is contacted for a period of from 0.5 to 100 h, or from 1 to 95 h, or from 6 to 90 h, or from 7 to 80 h, or from 8 to 70 h, or from 9 to 60 h, or from 10 to 50 h, or from 11 to 50 h, or from 12 to 40 h, or from 15 to 40 h, with an aqueous medium which is free from clostridial toxin.

In one embodiment, prior to said measuring in step (c) or step (h) or step (c) and step (h) and subsequent to the contacting in step (a) or step (f) or step (a) and step (f), the cell culture is lysed.

In another embodiment, the cell culture is lysed prior to the contacting in step (a) or step (f) or step (a) and step (f).

In one embodiment, said measuring is performed by Western-Blot analysis or ELISA.

In one embodiment, said cell culture is selected from cell cultures of neuronal cell lines or primary neuronal cells.

In one embodiment, said recording of said measured second effect is performed by plotting said second effect versus concentration, and said recording of said second data set is performed by recording a calibration curve.

In one embodiment, said second effect is determined at at least one concentration expressed in mouse LD₅₀ units/ml of at least 10.

In another embodiment, said concentration is from 10 to 1,000, or from 10 to 70, or from 15 to 60, or from 20 to 45.

In another embodiment, said concentration is from 20 to 400, or is from 100 to 800.

In one embodiment, said mouse LD₅₀ units are Xeomine® units.

In one embodiment, said clostridial neurotoxin is botulinum toxin.

In another embodiment, said botulinum neurotoxin is of a serotype selected from the group consisting of A, B, C, D, E, F and G; or is a chemically or genetically modified derivative of a botulinum neurotoxin of a serotype selected from the group consisting of A, B, C, D, E, F and G.

In another embodiment, said neurotoxin is of serotype A or C or E.

In one embodiment, the neurotoxin is free of complexing proteins.

In another aspect, the invention relates to a computer program product comprising a computer program comprising software means for implementing the method according to the invention.

In another aspect, the invention relates to the use of a cell culture in any one of the methods of the invention.

In another aspect, the invention relates to the use of the method of the invention according to any one of the first, second and third aspect of the invention for controlling the potency of a sample comprising a clostridial neurotoxin.

In one embodiment, the sample is a stored sample.

In one embodiment, the sample is a lyophilized sample or is a reconstituted sample.

In another aspect, the invention relates to the use of the method according to the first aspect of the invention for determining the unknown concentration of a clostridial neurotoxin in a first sample with respect to the known concentration of a clostridial neurotoxin in a second sample; or for determining the relative potency of a clostridial neurotoxin in a first sample with respect to the potency of a clostridial neurotoxin in a second sample, e.g. during the quality control during a process for the manufacture of clostridial neurotoxin.

Compared to the methods known from the prior art using cell cultures, the methods according to the invention allow for a significant improvement of accuracy and precision of the quantification of biological activity of clostridial botulinum neurotoxin. The methods according to the invention satisfy regulators requirements.

It has been found that the variability observed with the quantification methods of the prior art using cell cultures can be reduced significantly to an insignificant degree by applying the methods disclosed herein.

In one embodiment, the invention relates to a method for measuring an effect induced to a cell culture by a clostridial neurotoxin, comprising:
(a) contacting a cell culture with a sample comprising said clostridial neurotoxin;
(c) measuring an effect induced to said cell culture by said neurotoxin;

wherein step (c) is performed in the absence of said sample.

The term “contacting a cell culture with said sample (that may be a first or a second sample according to the methods according to the further aspects of the invention)” means that at least part of said neurotoxin of said sample is received by said cell culture during said contacting, i.e. at least part of the neurotoxin being contained in said sample is bound by appropriate receptors being contained in said cells of the cell cultures.

The term “absence of the sample” means that the measuring of the effect in step (c) is performed in a medium, typically an appropriate buffer, that contains 10% by weight or less, e.g. does not contain any, of the sample or, stated differently, of the neurotoxin of the sample.

In one embodiment, said cell culture is not continuously exposed to (contacted with) the sample (that may be a first or a second sample according to the methods according to the further aspects of the invention) comprising a clostridial neurotoxin, but only temporarily.

This means that after a predetermined period of exposing said cell culture to the neurotoxin, i.e. contacting in step (a) in order to effect a response of said cell culture to the exposure, the corresponding measurement of the effect (or a first, respectively second effect according to the methods according to the further aspects of the invention), is performed in the absence of said sample (that may be said first or said second sample according to the methods according to the further aspects of the invention) employing the methods as described below.

In one embodiment, prior to said measurement, said cell culture is e.g. removed from a bath containing said sample, and is transferred to a bath containing the neurotoxin-free ingredients as described below. Subsequently, the measurement of the magnitude of the effect (that may be a first or a second effect when the sample is a first or a second sample) is performed, i.e. the effect is quantified. This means that the response to said stimulation is performed with the cell culture containing the received neurotoxin.

In another embodiment, the neurotoxin-containing ingredients, i.e. the sample (that may be a first or a second sample), are replaced by neurotoxin-free ingredients. In one embodiment, the sample is removed from the cell culture by e.g. decanting and is replaced by neurotoxin-free ingredients as described below. Subsequent to the replacement, the measurement of the magnitude of the effect (that may be a first or a second effect when the sample is a first or a second sample) is performed.

The term “clostridial neurotoxin (or clostridial toxin)” encompasses clostridial toxin complexes as well as high purity neurotoxin, i.e. a neurotoxin preparation, which is free of any other clostridial proteins.

In one embodiment, said clostridial neurotoxin is botulinum neurotoxin.

In another embodiment, said botulinum neurotoxin is a serotype selected from the group consisting of A, B, C, D, E, F and G.

The term “botulinum toxin complex” encompasses a botulinum toxin associated with at least another non-toxic protein. As apparent, the term botulinum toxin complex, as used herein, comprises the 450 kDa and the 900 kDa botulinum toxin complex, which is e.g. obtainable from cultures of C. botulinum. Such preparations on the basis of botulinum toxin complex of type A are commercially available e.g. by Ipsen Ltd. (Dysport®) or Allergan Inc. (Botox®). Another preparation based on botulinum complex type B is available from Solstice Neurosciences, Inc. (Myobloc®). A high purity neurotoxin of type A, free of any other clostridial proteins is available from Merz Pharmaceuticals (Xeomin®). It is the drug of choice to improve several forms of focal dystonia.

In another embodiment, said botulinum neurotoxin is a chemically or genetically modified derivative of a serotype selected from the group consisting of A, B, C, D, E, F and G.

A chemically modified derivative of said neurotoxin may be one that is modified by pyruvation, phosphorylation, sulfatation, lipidation, and/or glycosilation.

A genetically modified derivative of said neurotoxin is one that has been modified by deletion, addition or substitution of one or more amino acids contained in the proteins of said serotype.

Such a modified toxin preferably is biologically active.

A biologically active toxin is a toxin being capable to be uptaken into a cell, thereby proteolytically cleaving one or more polypeptides such as SNAP25 involved in the SNARE complex. If the concentration of a proteolytically cleaved polypeptide such as SNAP25 is measured and quantified, the concentration or potency of the used toxin may be calculated.

In one embodiment according to any one of the methods according to the three aspects according to the invention, prior to said measuring in step (c) or step (h) or step (c) and step (h), said cell culture is exposed to (contacted with) said clostridial toxin for a period of from 5.0 to 45 h, or from 15 to 40 h, or from 25 to 35 h.

In another embodiment, prior to said measuring in step (c) or step (h) or step (c) and step (h) and subsequent to the contacting in step (a) or step (f) or step (a) and step (f), said cell culture is contacted for a period of from 0.5 to 100 h, or from 1 to 95 h, or from 6 to 90 h, or from 7 to 80 h, or from 8 to 70 h, or from 9 to 60 h, or from 10 to 50 h, or from 11 to 50 h, or from 12 to 40 h, or from 15 to 40 h, with an aqueous medium which is free from clostridial toxin.

The term “aqueous medium” defines a liquid or fluid comprising water.

In one embodiment, said aqueous medium is a buffer.

In one embodiment, said buffer is a neutral buffer. The term “neutral” encompasses a pH range of from 6 to 8, or from 6.5 to 7.5, or approx. 7.

In one embodiment, said buffer is a phosphate buffer.

In one embodiment, the temperature of said aqueous medium is from 20 to 40° C., or from 25 to 40° C., or from 30 to 40° C. In one embodiment, the temperature is approx. 37° C.

In one embodiment, prior to said measuring in step (c) or step (h) or step (c) and step (h) and subsequent to the contacting in step (a) or step (f) or step (a) and step (f), the cell culture is lysed.

The term “lysis” refers to the breaking down of a cell such as by viral, enzymatic or osmotic mechanisms that compromise its integrity. A fluid containing the contents of lysed cells is called a “lysate”. For example, lysis may be used in Western and Southern blotting to analyze the composition of specific proteins, lipids and nucleic acids individually or as complexes. For lysis, the commonly known lysis buffers may be used.

In another embodiment, the cell culture is lysed prior to the contacting in step (a) or step (f) or step (a) and step (f).

Surprisingly, it has been discovered that the measurement of said effect in the absence of said sample, and subsequent to the contacting with an aqueous medium which is free from a clostridial botulinum neurotoxin after said cell culture had been exposed to or contacted with the neurotoxin, shifts the respective dose-response curves such that the sensitivity of the method according to the invention is significantly increased. The sensitivity is particularly increased at low concentrations expressed in LD₅₀ mouse units/ml of said clostridial neurotoxin in said sample.

For example, if as effect, respectively response, the cleavage of a protein such as SNAP25 from the SNARE complex is determined, the method results in an advantageous increase of the sensitivity of the method, which in particularly applies in the region of lower concentrations of neurotoxin. If the potency is determined at lower concentration, neurotoxins in general may exhibit the greatest variances, whereas at rather high concentrations potencies converge to each other.

This increasing of the sensitivity allows for a more precise and more reliable analysis of the respective dose-response curves. This in turn allows for a considerably lower amount of laboratory animals such as mice, which otherwise have to be sacrificed in order to perform any one of the methods according to the invention. Accordingly, this embodiment of the invention is not only a progress under technical aspects but also under ethical aspects.

The term “sensitivity” is used herein in the meaning as commonly used in physiology, i.e., it defines the ability of a cell culture to respond to an external stimuli. Here, the external stimuli is performed by contacting a cell culture with a clostridial neurotoxin. It is within the ambit of the invention that a certain concentration range may be chosen, such as a concentration range at relatively low concentration of clostridial neurotoxin, where said sensitivity is increased, i.e. a response can be determined that otherwise can not be determined, respectively can only determined within a non-tolerable deviation.

In another embodiment, the invention relates to a method for determining the unknown concentration of a clostridial neurotoxin in a first sample with respect to the known concentration of a clostridial neurotoxin in a second sample, the method comprising:
(a) contacting a cell culture with said second sample;
(c) measuring a second effect induced to said cell culture by said neurotoxin;
(d) repeating steps (a) to (c) at various concentrations of said clostridial neurotoxin;
(e) recording said measured second effect of step (d) versus concentration, thereby recording a second data set;
(f) contacting a cell culture with said first sample;
(h) measuring a first effect induced to said cell culture;
(k) identifying the concentration for which said first and said second effect are identical;
(l) equating said concentration in (k) to said unknown concentration.
wherein step (c) and/or step (h) is/are performed in the absence of said second and/or first sample.

Accordingly, in one embodiment, the determination of the second and/or the first effect is performed in the absence of said second and/or first sample. This means that after step (a) and/or after step (f) said cell culture is removed from the second and/or the first sample, respectively the second and/or first sample are removed from the cell culture as disclosed above.

The term “identifying the concentration for which said first and said second effect are identical” (steps (k) and (l)) means that said first and second effect are qualitatively and quantitatively identical, i.e. the induced effect is e.g. the cleavage of a protein or polypeptide such as SNAP25 from a SNARE complex, and that said effects have the same measured value.

In one embodiment, in order to obtain results that can reliably be compared, the exposure time of the cell culture to the neurotoxin being contained in the second, respectively the first sample, should be comparable.

In one embodiment, said exposure times are identical.

In one embodiment, said recording of said measured second effect in step (e) is performed by measuring said second effect at various concentrations of said clostridial neurotoxin in said second sample and plotting said measured second effect versus concentration, thereby recording a calibration curve.

If the effect induced by said second sample to said cell culture is determined on the basis of various concentrations expressed in mouse LD₅₀ units/ml, a calibration curve may be obtained, as described above.

For example, it is possible, to determine said effect induced in steps of ten LD₅₀ units/ml or of five LD₅₀ units/ml within a selected concentration range.

Accordingly, by means of the second data set recorded in step (e), a calibration curve is plotted by means of which the unknown concentration of said clostridial neurotoxin in said first sample is identified according to steps (k) and subsequent step (l).

In one embodiment, the generated calibration curve is plotted, and said steps of identifying and equating according to steps (k) to (l), are performed by a graphic analysis.

Said unknown concentration of the first sample can be determined by identifying the concentration from the calibration curve for which said first and said second effect have the same value, e.g. the same concentration of produced SNAP25, and equating said concentration to said unknown concentration according to step (l).

A prerequisite for said determination is that the unknown concentration of the clostridial toxin in the first sample exerts an effect on the cell culture, which can be quantified by means of said calibration curve. The person skilled in the art will readily acknowledge that it may be necessary to dilute or concentrate the first sample having the unknown concentration once or several times if necessary in order to achieve a concentration range, wherein a comparison with the second sample is possible, i.e. to achieve identical first and second effects. Then, knowing the dilution or the concentration factor, the calculation of the concentration of the neurotoxin being originally present in the not diluted or not concentrated first sample may be determined.

In another embodiment, said identification and equation is not performed by a single-point measurement of only one concentration in step (h) and subsequent steps (k) and (l), but by measurement at a multitude of various concentrations. This is particularly important in view of regulatory requirements.

According to another embodiment of the invention, it is desirable to optimize the concentration range in which a reliable comparison of said second and first sample is possible. This does not only apply to the comparability regarding the biological efficacy of hitherto known and commercial formulations of clostridial neurotoxins, but also to formulations, which might by developed in future or being already under development.

In one embodiment, in order to optimize the concentration range expressed in mouse LD₅₀ units/ml in which a reliable comparison of said second and first sample is possible, it is desirable to firstly determine the standard deviation of the calibration curve recorded in step (e) and/or in step (h). By using a suitable step-wise regression analysis, it is possible to generate a regression model for predicting the potency of an unknown toxin sample based on the dose-response curve.

By means of such method, it is possible to identify a concentration range for the first and the second sample representing two different data populations, in which the correlation between the respective dose-response curves reaches a maximum, i.e. the best fit is determined.

In one embodiment, the test may be further refined by representing a value range of the respective data sets of the first and the second sample by fit curves according to a predetermined regression model, respectively, and linearizing and parallelizing said fit curves within a predetermined confidence interval.

Accordingly, according to a third aspect, the invention relates to a method of determining the relative potency of a clostridial neurotoxin in a first sample with respect to the potency of clostridial neurotoxin in a second sample, the method comprising:
(a) contacting a cell culture with said second sample;
(c) measuring a second effect induced to said cell culture by said neurotoxin;
(d) repeating steps (a) to (c) at various concentrations of said clostridial neurotoxin;
(e) recording said measured second effect of step (d) versus concentration, thereby recording a second data set;
(f) contacting a cell culture with said first sample;
(h) measuring a first effect induced to said cell culture obtained in step (g);
(i) repeating steps (f) to (h) at various concentrations of said clostridial neurotoxin;
(j) recording said measured first effect of step (i) versus concentration, thereby recording a first data set;
wherein step (c) and/or step (h) is/are performed in the absence of said second and/or first sample.

In one embodiment, the method further comprises steps (m) and (n):
(m) selecting said various concentrations from a concentration range that best fits to the first and the second data set;
(n) determining said best fit by a statistical test comprising the following sub-steps (α) to (δ):
   (α) representing a value range of the second data set obtained in step (e) by a fit curve;
   (β) representing a value range of the first data set obtained in step (j) by a fit curve;
   (γ) linearizing the fit curves, respectively;
   (δ) parallelizing the linearized fit curves.

In one embodiment, the determination of the second and/or the first effect is performed in the absence of said second and/or first sample.

In another embodiment, the measurement of the effect is performed in the absence of said second and/or first sample. This means that after step (a) and/or after step (f) said cell culture is removed from the second and/or the first sample as disclosed above, or the second and/or the first sample are removed from the cell culture.

Statistical tests suitable for performing the above sequence are well known, such as likelihood-quotient-tests. An example of such a likelihood-quotient-test is the known F-test. Test such as the x²-Test (chi-squared-test or x²-distribution-test) or the t-test may also be employed. Said tests are also known in the art.

In one embodiment, said statistical test is the F-test.

By means of said test, it is possible to decide within a predetermined confidence interval whether two random samples taken from two different populations essentially differ with respect to the variance thereof. Therefore, such a test serves for the testing of differences within two statistical samples, here the second and the first sample.

In one embodiment, the confidence interval should be broad in order to obtain reliable results, i.e. the false-rejection probability should be relatively low.

In one embodiment, the false-rejection probability is 5 (expressed in %; (or 0.05)), respectively the confidence interval is ≧95 (expressed in %; (or 0.95)).

In one embodiment, the false-rejection probability for each sub-step (α) to (δ) is ≦5 (expressed in %).

In one embodiment, linearizing in step (γ) is performed by representing the respective data sets by a best fit straight line.

In one embodiment, parallelizing in step (δ) is performed by determining a common slope of the best fit straight lines.

Subsequent to step (δ), from the shift of the linearized and parallelized fit curves relative to each other, the relative potency of the first sample versus the second sample is determined.

Accordingly, in one embodiment, the method further comprises after step (δ) step (ε):
(ε) calculating from the shift of the linearized and parallelized fit curves relative to each other the relative potency of the first sample with respect to the second sample.

In one embodiment, the term “relative potency” means that the potency of the first sample with respect to the second sample is determined at identical concentration, respectively identical concentrations, from the respective linearized and parallellized fit curves.

In one embodiment, the potency of the second sample is equated to 100%, and the relative potency of the first sample is expressed in terms of %. E.g., one obtains for the first sample a potency of e.g. 110% or 90% with respect to the second sample. By respective dilution of the first sample having the 110% potency to the 100% potency, one obtains the effective concentration of the clostridial neurotoxin in the first sample, which hitherto was not known, by application of the rule of three. The unit for measurement now becomes relative potency, and the value is expressed as a unit of activity (potency) defined in terms of the activity (potency) of the reference standard (second sample).

In another embodiment, the relative potency is expressed as ratio of the potency of the first and the second sample.

In one embodiment, the above described model is used to predict the logarithmic value of the applied neurotoxin dose.

In another embodiment, both the quantity of the stimulated effect and the quantity of the neurotoxin dose in the sample are recorded in a logarithmic scale.

In one embodiment, the second effect, respectively the first effect, are measured at at least three different concentrations of the clostridial neurotoxin in the second sample, respectively the first sample.

In one embodiment, said recording of said date sets, respectively said recording of a calibration curve, respectively calibration curves, is performed in the form of a semi logarithmic plot.

In another embodiment, a double logarithmic plot is performed.

The method of determining a relative potency is documented in the European Pharmacopoeia.

In one embodiment, starting with a concentration of e.g. 10 mouse LD₅₀ units/ml, the method of determining said relative potency is applied over the whole range of the data set. Subsequently, values greater than 10 mouse LD₅₀ units/ml are used as starting points, such as 11, 12, 13, 14, 15, 16, 17 mouse LD₅₀ units/ml. Said iteration is performed as long until the applied model yields the desired and required accuracy.

In one embodiment, once a best fit and thus a concentration range has been identified by the statistical test, any first sample having an unknown concentration (with regard to the effective concentration) of a clostridial neurotoxin may be compared with respect to the known concentration of said clostridial neurotoxin in a second sample within said concentration range identified according to the method of the invention.

In one embodiment, said recording of said measured second effect is performed by plotting said second effect versus concentration, and said recording of said second data set is performed by recording a calibration curve.

The use of relative potency estimates, and the inclusion of a reference standard (second sample) in the assay, lead to more precise and more reproducible estimates, which provide opportunities for reductions in animal use.

Statistical tests are commonly performed by means of a suitable computer program and a suitable computer.

In one embodiment, the statistical test is performed by means of a suitable computer program comprising suitable software means for implementing the statistical test.

Accordingly, in one embodiment, the invention relates to a computer program product comprising a computer program comprising software means for implementing the method according to the invention.

In one embodiment, the second sample is selected from a commercially available and registered botulinum toxin preparation. Since these products are registered and allowed as a pharmaceutical preparation, respectively medicament, they comprise a clearly defined quantity, respectively concentration of a botulinum toxin.

In another embodiment, any botulinum toxin preparation may be used that has been produced under standard conditions.

In one embodiment, the commercial preparations mentioned above may be used as the second sample. Thus, the second sample may be Xeomin®, Botox®, Dysport®, Myobloc® or PurTox®. These preparations either differ in the used botulinum toxin type or in biological efficacy/activity, i.e. potency, e.g. in the concentration of the botulinum neurotoxin or in the botulinum type contained therein.

The mouse unit expressed in terms of mouse LD₅₀ is a commonly accepted unit to define a concentration of a clostridial neurotoxin contained in a sample. The LD₅₀ value defines the lethal dose at which 50% of a mouse population is killed if said quantity is applied to the mice of said mouse population. The method for determining said value is known to the person skilled in the art. Such method is documented in the European Pharmacopoeia.

As is known, the LD₅₀ units in the labeling of the products based on a botulinum neurotoxin may be product-specific, respectively manufacturer-specific, and may be non-interchangeable due to the absence of a standard.

In one embodiment, the LD₅₀ units referred to herein are units as determined in the characterization and labeling of Xeomin®. E.g., the second sample is Xeomin®. Accordingly, the units relating to a certain potency are Xeomin® units. Therefore, the assay system of the present invention can be used for comparably assessing the potency of any sample comprising a clostridial neurotoxin relative to Xeomin®. Then, the method allows to directly compare first samples comprising a clostridial neurotoxin (in an unknown concentration) in terms of Xeomin® units.

Xeomin® and Botox® exhibit an approximately comparable efficacy or potency. In order to obtain the same efficacy or potency as Xeomin® and Botox®, approximately the 2.5-fold quantity of Dysport®, respectively the 10-fold quantity of Myobloc® have to be applied.

In one embodiment, these commercially available preparations are diluted or concentrated to predetermined concentrations of the botulinum neurotoxin contained therein, and said second effect is measured in dependence of various concentrations of said clostridial neurotoxin in said second sample. Said measured effect is plotted versus concentration of botulinum toxin, thereby recording a calibration curve. By means of said second data set, respectively said calibration curve, the unknown concentration of botulinum neurotoxin in a first example may be determined.

It has been discovered that a concentration of a clostridial neurotoxin in a sample (that may be a first or a second sample) expressed in mouse LD₅₀ units/ml of at least 10, the methods according to the invention can be advantageously applied. It is to be noted that the concentration given within the present application are all mouse LD₅₀ units/ml.

In one embodiment, the sample comprises besides the neurotoxin water. In one embodiment, the sample comprises a solution or suspension of the neurotoxin in water.

In one embodiment, said concentration of the neurotoxin in said sample is at least 15.

In another embodiment, said concentration is at least 20.

In another embodiment, said concentration is from 10 to 1,000.

In one embodiment, the concentration is from 10 to 70.

In another embodiment, the concentration is from 15 to 60.

In still another embodiment, the concentration is from 20 to 45.

In one embodiment, the second sample is Xeomin®.

In one embodiment, it has been discovered that if Xeomin® is used as the second sample, particularly reliable results are obtained, if the second effect is determined at at least one concentration of from 10 to 70. In another embodiment, the concentration is from 15 to 60. In still another embodiment, the concentration is from 25 to 45.

In one embodiment, it has been discovered that if Botox® is used as the second sample, reliable results are obtained, if the second effect is determined at at least one concentration of from 10 to 70. In another embodiment, the concentration is from 15 to 60. In still another embodiment, the concentration is from 25 to 45.

If a second sample is used for determining the calibration curve according to step (e), the second sample having a lower concentration or comprising a less efficient or potent botulinum neurotoxin than Xeomin® or Botox®, higher concentrations of the neurotoxin, i.e. higher LD₅₀ units/ml values are required in order to achieve a strength of the second effect that is comparable to the effect induced by Xeomin® or Botox®.

In an embodiment, wherein the second sample has a lower concentration or potency of botulinum neurotoxin than Xeomin® or Botox®, the second effect is determined at at least one concentration of from 20 to 400, or from 100 to 800.

In one embodiment, wherein the second sample is Dysport®, the second effect is determined at at least one concentration of from 20 to 400, or from 25 to 300, or from 30 to 250.

In another embodiment, wherein the second sample is Myobloc®, the second effect is determined at at least one concentration of from 100 to 800, or from 150 to 700, or from 200 to 600.

In other embodiments, the concentration may range from 30 to 600, or 30 to 400, or 30 to 200, or 30 to 100, or 30 to 80, or 40 to 500, or 40 to 400, or 40 to 300, or 40 to 200, or 40 to 100, or 40 to 90, or 50 to 300, or 50 to 200, or 50 to 100, or 60 to 100, depending on the concentration of the efficacy or potency of the neurotoxin in the second sample compared to Xeomin® or Botox®.

In one embodiment, the LD₅₀ units are Xeomin® units.

In one embodiment, due to the reliability of said cell culture assay, it is possible to comply with certification requirements of regulatory authorities and to satisfy the need for a safe and effective administration of botulinum toxin such as of serotype A or serotype C or serotype E.

In still another embodiment, said clostridial toxin in said first sample and said clostridial toxin in said second sample are the same clostridial toxins.

In still another embodiment, said clostridial toxin or neurotoxin in the first sample and said clostridial toxin or neurotoxin in said second sample are different from each other.

For the experimental realization of the method, typically a cell culture is used, which responds to the exposure to a botulinum toxin, i.e. the botulinum toxin exerts an effect on the cell culture such as the cleavage of a protein or polypeptide in a SNARE complex.

The term “cell culture” encompasses cells which are grown under controlled conditions outside of an organism.

In one embodiment, the term “cell culture” refers to the culturing of cells derived from multicellular eukaryotes, especially animal cells. However, the term also encompasses cell cultures of plants, fungi and microbes, including viruses, bacteria and protists.

The methods of culturing cells are well known in the art. In one embodiment, cells may be isolated from tissues for ex vivo culture. In one embodiment, pieces of tissue can be placed in growth media, and the cells that grow out are available for culture. In another embodiment, cells may be purified from soft tissues by enzymatic digestion with enzymes such as collagenase, trypsin, or pronase, which break down the extracellular matrix. If immortalized cell lines are employed, such cell lines often have the ability to proliferate indefinitely either through random mutation or deliberate modification. Cells can be grown in suspension or adherent cultures. Depending on the cell type, cells may naturally live in suspension without being attached to a surface. Adherent cells require a surface, such as tissue culture plastic or micocarrier, which may be coated with extracellular matrix components to increase adhesion properties and provide other signals needed for growth and differentiation.

In one embodiment, for the experimental realization of the method according to the invention, cells may be grown and maintained at an appropriate temperature and gas mixture, e.g. at 37° C., and 5% CO₂ in a cell incubator. Culture conditions may vary widely for each cell type, and variation of conditions for a particular cell type may result in different phenotypes being expressed. Aside from temperature and gas mixture, the most commonly varied factor in culture systems is the growth medium. Recipes for growth media may vary in pH, glucose concentration, growth factors, and the presence of other nutrients, and the like. The person skilled in the art is familiar with said various kinds of culturing cells.

After harvesting, the cultured cells may be employed in any one of the methods according to the invention.

In one embodiment, the cells are selected from neuronal cell lines or primary neuronal cell cultures.

The term “cell line” encompasses cells of one type, which proliferate indefinitively.

The term “primary cells” encompasses a non-immortalized cell line, which was directly obtained from a tissue.

In one embodiment, the cells of the cell culture comprise spinal cord cells.

In one embodiment, the cells, e.g. the spinal cord cells, of the cell culture are obtained from a rodent. In one embodiment, the cells of the cell culture are mouse spinal cord cells or rat spinal cord cells.

In one embodiment, cell cultures as used in the prior art section (see Pellet, S. et al; Keller, J. E. et al) may be employed for the purpose of the invention.

According to one aspect, the invention also provides an improved method of identifying a concentration range in which in which the potency of a first sample comprising a clostridial neurotoxin relative to a second sample comprising a clostridial neurotoxin can be determined within a predetermined confidence interval or false-rejection probability.

In one embodiment, such method of identifying a concentration range in which the potency of a first sample comprising a clostridial neurotoxin relative to a second sample comprising a clostridial neurotoxin may be determined, comprises the following steps:
(a) contacting a cell culture with said second sample;
(c) measuring a second effect induced to said cell culture by said neurotoxin;
(d) repeating steps (a) to (c) at various concentrations of said clostridial neurotoxin;
(e) recording said measured second effect of step (d) versus concentration, thereby recording a second data set;
(f) contacting a cell culture with said first sample;
(h) measuring a first effect induced to said cell culture by said neurotoxin;
(i) repeating steps (f) to (h) at various concentrations of said clsotridial neurotoxin;
(j) recording said measured first effect of step (i) versus concentration, thereby recording a first data set;
wherein said concentration is selected from a concentration range that best fits to the first and the second data set, and wherein said best fit is determined by a statistical test comprising the following sub-steps (α) to (δ):
(α) representing a value range of the second data set obtained in step (e) by a fit curve;
(β) representing a value range of the first data set obtained in step (j) by a fit curve;
(γ) linearizing the fit curves, respectively;
(δ) parallelizing the linearized fit curves.

In said embodiment, said second and said first effect are qualitatively identical. For refining the method, the methods as described above in connection with the method according to the third aspect of the invention can be used.

In a further aspect of the invention, the methods of the invention may be advantageously used for controlling the quality, i.e. the potency of a sample comprising a clostridial neurotoxin with respect to a reference standard such as is required in a manufacturing process.

Accordingly, in said aspect, the invention relates to the use of the method of the invention for controlling the quality, i.e. the potency of a sample comprising a clostridial neurotoxin.

In one embodiment, the potency of a sample is determined that has been stored. In one embodiment, the sample has been stored for a period of at least one hour, or at least one day.

In one embodiment, the sample is a lyophilized sample, or is a reconstituted sample.

According to another aspect, the invention relates to the use of the method according to the first aspect of the invention for determining the unknown concentration of a clostridial neurotoxin in a first sample with respect to the known concentration of a clostridial neurotoxin in a second sample; or for determining the relative potency of a clostridial neurotoxin in a first sample with respect to the potency of a clostridial neurotoxin in a second sample.

According to a further aspect, the invention relates to the use of a cell culture, in particular a cell culture comprising spinal cord cells, such as cells from rat or mouse, for determining clostridial activity in any one of the methods of the invention.

The following embodiments also belong to the invention and are to be understood that the embodiments described above apply vice versa to the methods listed below.

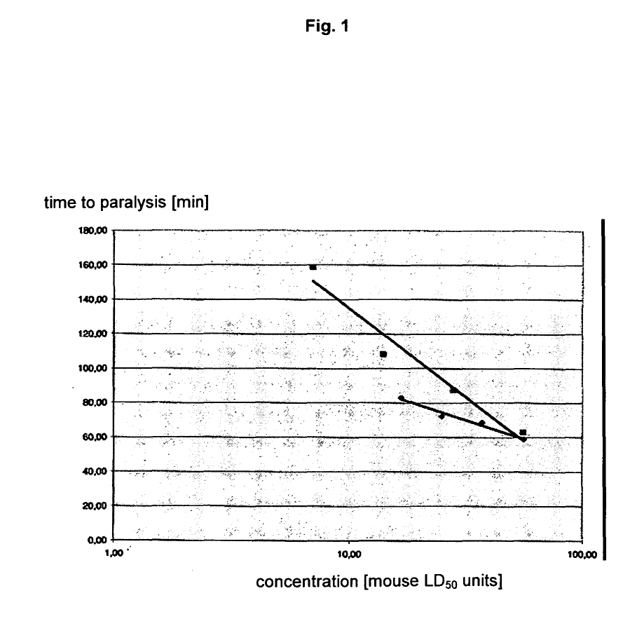

FIG. 1 shows a plot of the time to paralysis (time needed to reach half the initial contraction force of a hemidiaphragm) expressed in minutes versus the concentration of botulinum neurotoxin NT expressed in mouse LD₅₀ units applied to an organ bath (half logarithmic scale). Curve ▪ represents a sample, wherein the induced effect is measured in the presence of the neurotoxin, and curve ♦ represents the sample, wherein the tissue has been exposed to the sample containing neurotoxin for a period of 15 minutes. Subsequently, the muscle tissue was removed from the bath, and the sample was replaced by a ingredients being free from neurotoxin. After performing the electrical stimulation, the induced effect was measured. The curves represent fit lines determined according to the method of the invention.

### Example 1

For a standard measurement, a mouse hemidiaphragm was prepared and applied to an organ bath filled with Earle's Balanced Salt Solution. The nervus phrenicus of the hemidiaphragm was mounted to a platinum electrode by which the nerve was electrically stimulated, subsequently effecting the contraction of the hemidiaphragm. The hemidiaphragm was clamped in the organ bath. During the clamping, the stimulation was switched off, however immediately switched on after the clamping. The intensity of the electrical current for stimulation was selected such that a contraction force of the hemidiaphragm could be measured. After a constant contraction force could be measured, the medium was exchanged against medium containing botulinum neurotoxin. The time needed to reach half the contraction force (paralysis time) was determined for each concentration (at least for times per concentration) and was plotted against the concentration of botulinum neurotoxin applied to the organ bath.

1-53. (canceled)

54. A method of measuring an effect induced by a clostridial neurotoxin in a cell culture or a muscle tissue, comprising:
(a) contacting a cell culture or a muscle tissue with a sample comprising the clostridial neurotoxin;
(c) measuring the effect of the clostridial neurotoxin on the cell culture or the muscle tissue;
wherein step (c) is performed in the absence of the sample, and wherein the cell culture is contacted for a period of from 0.5 to 100 hours (hr) with an aqueous medium which is free from a clostridial toxin prior to the measuring in step (c) and subsequent to the contacting in step (a).

55. The method of claim 54, wherein prior to the measuring in step (c) and subsequent to the contacting in step (a), the cell culture is contacted with the sample comprising the clostridial toxin for a period of from 5 to 45 hr, from 15 to 40 hr, from 25 to 35 hr, from 1 to 95 hr, from 6 to 90 hr, from 7 to 80 hr, from 8 to 70 hr, from 9 to 60 hr, from 10 to 50 hr, from 11 to 50 hr, from 12 to 40 hr, or from 15 to 40 hr, with an aqueous medium which is free from a clostridial toxin.

56. The method of claim 54, wherein subsequent to contacting the muscle tissue with a sample comprising the clostridial neurotoxin in step (a), the method further comprises a step of:
(b) electrically stimulating the muscle tissue obtained in step (a).

57. The method of claim 56, wherein step (b) is performed in the absence of a sample comprising the clostridial neurotoxin.

58. The method of claim 54, wherein the effect is cleavage of a protein from a SNARE complex.

59. The method of claim 54, wherein the muscle tissue is selected from intercostal muscle, hind limb muscle, the hind limb extensor digitorum longus muscle, the plantar muscles of the hind paw, the phrenic nerve-hemidiaphragm, the levator auris longus muscle, the frog neuromuscular junction, the biventer cervic muscle of chicks, rib muscles, brain tissue and the electrical organ of the sea ray.

60. The method of claim 54 wherein an unknown concentration of a clostridial neurotoxin in a first sample with respect to the known concentration of a clostridial neurotoxin in a second sample, or wherein the relative potency of a clostridial neurotoxin in a first sample with respect to the potency of a clostridial neurotoxin in a second sample is determined.

61. A method of determining an unknown concentration of a clostridial neurotoxin in a first sample with respect to a known concentration of a clostridial neurotoxin in a second sample, the method comprising:
(a) contacting a cell culture or a muscle tissue with the second sample;
(c) measuring a second effect induced in the cell culture or the muscle tissue by the neurotoxin;
(d) repeating steps (a) to (c) at various concentrations of the clostridial neurotoxin;
(e) recording the measured second effect of step (d) versus concentration, thereby recording a second data set;
(f) contacting a cell culture or a muscle tissue with the first sample;
(h) measuring a first effect induced in the cell culture or the muscle tissue;
(k) identifying the concentration for which the first and the second effect are identical; and
(l) equating the concentration in (k) to the unknown concentration;
wherein step (c) and/or step (h) is/are performed in the absence of the second and/or first sample, and wherein the cell culture is contacted for a period of from 0.5 to 100 hours with an aqueous medium which is free from a clostridial toxin prior to the measuring in step (c), or step (h), or step (c) and step (h), and subsequent to the contacting in step (a), or step (f), or step (a) and step (f).

62. The method of claim 61, wherein prior to the measuring in step (c), or step (h), or step (c) and step (h), the cell culture is contacted with the sample comprising the clostridial toxin for a period of from 5 to 45 hr, from 15 to 40 hr, from 25 to 35 hr, from 1 to 95 hr, from 6 to 90 hr, from 7 to 80 hr, from 8 to 70 hr, from 9 to 60 hr, from 10 to 50 hr, from 11 to 50 hr, from 12 to 40 hr, or from 15 to 40 hr, with an aqueous medium which is free from a clostridial toxin.

63. The method of claim 61, wherein subsequent to contacting the muscle tissue with a sample comprising the clostridial neurotoxin in step (a), the method further comprises the step of:
(b) electrically stimulating the muscle tissue obtained in step (a); and/or wherein subsequent to contacting the muscle tissue with a sample comprising the clostridial neurotoxin in step (f), the method further comprises the step of:
(g) electrically stimulating the muscle tissue obtained in step (f).

64. The method of claim 63, wherein steps (b) or (g) are performed in the absence of the second or the first sample, or wherein steps (b) and (g) are performed in the absence of the second and the first sample.

65. The method of claim 61, wherein the recording of the measured second effect is performed by plotting the second effect versus concentration, and the recording of the second data set is performed by recording a calibration curve.

66. The method of claim 61, wherein the first and second effects are selected from time to paralysis of the muscle tissue, variation in the contraction rate of the muscle tissue, variation in the contraction distance of the muscle tissue, variation in the force of contraction of the muscle tissue, variation in the end plate potential and the miniature end plate potential of the muscle tissue.

67. The method of claim 61, wherein the effect is cleavage of a protein from a SNARE complex.

68. The method of claim 61, wherein the muscle tissue is selected from intercostal muscle, hind limb muscle, the hind limb extensor digitorum longus muscle, the plantar muscles of the hind paw, the phrenic nerve-hemidiaphragm, the levator auris long us muscle, the frog neuromuscular junction, the biventer cervic muscle of chicks, rib muscles, brain tissue and the electrical organ of the sea ray.

69. A method of determining the relative potency of a clostridial neurotoxin in a first sample with respect to the potency of clostridial neurotoxin in a second sample, the method comprising:
(a) contacting a cell culture or a muscle tissue with the second sample;
(c) measuring a second effect induced in the cell culture or the muscle tissue by the neurotoxin;
(d) repeating steps (a) to (c) at various concentrations of the clostridial neurotoxin;
(e) recording the measured second effect of step (d) versus concentration, thereby recording a second data set;
(f) contacting a cell culture or a muscle tissue with the first sample;
(h) measuring a first effect induced in the cell culture or the muscle tissue;
(i) repeating steps (f) to (h) at various concentrations of the clostridial neurotoxin;
(j) recording the measured first effect of step (i) versus concentration, thereby recording a first data set;
wherein step (c) and/or step (h) is/are performed in the absence of the second and/or first sample, and wherein the cell culture is contacted for a period of from 0.5 to 100 hours with an aqueous medium which is free from clostridial toxin prior to the measuring in step (c), or step (h), or step (c) and step (h), and subsequent to the contacting in step (a), or step (f), or step (a) and step (f).

70. The method of claim 69, wherein subsequent to step (j) the method further comprises the steps of:
(k) selecting the various concentrations from a concentration range that best fits to the first and the second data set;
(l) determining the best fit by a statistical test comprising the following sub-steps (α) to (δ):
(α) representing a value range of the second data set obtained in step (e) by a fit curve;
(β) representing a value range of the first data set obtained in step (j) by a fit curve;
(γ) linearizing the fit curves, respectively; and
(δ) parallelizing the linearized fit curves.

71. The method of claim 70, wherein subsequent to step (δ) the method further comprises the step of:
(ε) calculating from the shift of the linearized and parallelized fit curves relative to each other the relative potency of the first sample with respect to the second sample.

72. The method of claim 69, wherein prior to the measuring in step (c), or step (h), or step (c) and step (h), the cell culture is contacted with the sample comprising the clostridial toxin for a period of from 5 to 45 hr, from 15 to 40 hr, from 25 to 35 hr, from 1 to 95 hr, from 6 to 90 hr, from 7 to 80 hr, from 8 to 70 hr, from 9 to 60 hr, from 10 to 50 hr, from 11 to 50 hr, from 12 to 40 hr, or from 15 to 40 hr, with an aqueous medium which is free from a clostridial toxin.

73. The method of claim 69, wherein subsequent to contacting the muscle tissue with a sample comprising the clostridial neurotoxin in step (a), the method further comprises the step of:
(b) electrically stimulating the muscle tissue obtained in step (a); and/or wherein subsequent to contacting the muscle tissue with a sample comprising the clostridial neurotoxin in step (f), the method further comprises the step of:
(g) electrically stimulating the muscle tissue obtained in step (f).

74. The method of claim 73, wherein steps (b) or (g) are performed in the absence of the second or the first sample, or wherein steps (b) and (g) are performed in the absence of the second and the first sample.

75. The method of claim 69, wherein the recording of the measured second effect is performed by plotting the second effect versus concentration, and the recording of the second data set is performed by recording a calibration curve.

76. The method of claim 69, wherein the effect is cleavage of a protein from a SNARE complex.

77. The method of claim 69, wherein the first and second effects are selected from time to paralysis of the muscle tissue, variation in the contraction rate of the muscle tissue, variation in the contraction distance of the muscle tissue, variation in the force of contraction of the muscle tissue, variation in the end plate potential and the miniature end plate potential of the muscle tissue.

78. The method of claim 69, wherein the muscle tissue is selected from intercostal muscle, hind limb muscle, the hind limb extensor digitorum longus muscle, the plantar muscles of the hind paw, the phrenic nerve-hemidiaphragm, the levator auris longus muscle, the frog neuromuscular junction, the biventer cervic muscle of chicks, rib muscles, brain tissue and the electrical organ of the sea ray.

79. A computer program product comprising, a computer program comprising software means for implementing the method of claim 69.
